(19) United States
(12) Patent Application Publication
(10) Pub. No.: US 2010/0305551 A1
(43) Pub. Date: Dec. 2, 2010

(54) Valveless Impedance Pump Drug Delivery Systems

(75) Inventors: Thomas J. Lobl, Valencia, CA (US); Jacob E. Pananen, Pasadena, CA (US); David L. Canfield, Lake Hughes, CA (US); Anna I. Nagy, Saugus, CA (US); Adrian L. Krag, Santa Clarit, CA (US)

Correspondence Address: BANNER & WITCOFF, LTD., 1100 13th STREET, N.W., SUITE 1200, WASHINGTON, DC, 20005-4051, US

(73) Assignee: NEUROSYSTEC CORP., Valencia, CA (US)

(21) Appl. No.: 12/793,731

(22) Filed: Jun. 4, 2010

Related U.S. Application Data
(63) Continuation of application No. 12/355,868, filed on Jan. 19, 2009, now abandoned.
(60) Provisional application No. 61/022,224, filed on Jan. 18, 2008.
(60) Provisional application No. 61/055,735, filed on May 23, 2008.
(60) Provisional application No. 61/077,843, filed on Jul. 2, 2008.

(51) Int. Cl. A61M 5/142

(52) U.S. Cl. 604/891.1

(57) ABSTRACT

A drug-delivery unit suitable for implantation into a patient body may include a valveless impedance pump. In some implementations the unit may include an actuator, control electronics and a battery, and may communicate with an external patient interface unit. The patient interface unit can be used to control operation of the implant and to download data from the implant. The patient interface unit can also be used to charge the implant and/or a separate charger can be used. In other implementations, a drug-delivery implant unit may lack internal electronics and instead rely on an externally-supplied magnetic field to actuate the pump.

### CROSS-REFERENCE TO RELATED APPLICATIONS

This application is a continuation of U.S. patent application Ser. No. 12/355,868 (filed Jan. 19, 2009, and titled “Valveless Impedance Pump Drug Delivery Systems”), which application claims the benefit of U.S. Provisional Application Ser. No. 61/022,224 (filed Jan. 18, 2008, and titled “Implantable Drug Delivery Systems Having Valveless Impedance Pumps, and Methods of Using Same”), U.S. Provisional Application Ser. No. 61/055,735 (filed May 23, 2008, and titled “Fluid Pumping System”) and U.S. Provisional Application Ser. No. 61/077,843 (filed Jul. 2, 2008, and titled “High Voltage/Low Current Output Circuits; Fluid Pumping Systems and Generating Voltages for Same”). The contents of all of these applications are incorporated by reference herein.

### BACKGROUND

It is known that drugs work optimally in the human body if they are delivered locally, e.g., to a specific tissue to be treated. When a drug is delivered systemically, tissues other than those being treated may be exposed to large quantities of that drug. This exposure presents a much greater chance for side effects. Targeting drug delivery to specific tissue often presents challenges, particularly if the targeted tissues are deep inside the body or are protected by a barrier to larger drug molecules. These challenges may be exacerbated if a drug must be delivered in multiple doses, over a prolonged period, to a location that can only be reached by an invasive medical procedure.

### SUMMARY

This Summary is provided to introduce a selection of concepts in a simplified form that are further described below in the Detailed Description. This Summary is not intended to identify key features or essential features of the invention.

At least some embodiments include an implant unit having a valveless impedance pump. Such an implant unit can be implanted into the body of a patient and used, in conjunction with an appropriate terminal component, to deliver small amounts of drug to a target tissue over a prolonged period. In some embodiments, an implant unit can include (or be used in combination with) a drug reservoir containing a solid drug that is removable by fluid flow generated by the valveless impedance pump. In some embodiments, the implant unit may also contain control electronics, an actuator, a battery and a coil usable to communicate with an external device and to generate power for recharging the battery. The actuator may include an electromagnet or a piezoelectric element. In certain embodiments, an implant unit lacks internal electronics and instead relies on an externally-provided magnetic field to move a force-transferring member of the valveless impedance pump.

Various embodiments also include a patient interface unit configured to communicate with an implant unit after the implant unit has been implanted into a patient's body. The patient interface unit can be used to activate and deactivate an implant unit, to transfer programming instructions to the implant unit (e.g., to set a time and/or a duration of pump activation), and to download data from an implant unit. In some embodiments, a patient interface unit can be used to charge an implant unit using a magnetic coil used for communication with the implant unit. A separate charging unit could also (or alternatively) be provided. An implant unit may in some embodiments be configured to communicate with physician interface software executing on a PC or other computer. Using such software, a physician or other user could download data from the patient interface unit and use such data to track dosage history of drug delivered with the implant unit. Such software could also be used to program the patient interface unit so as to limit the manner in which a patient could utilize the patient interface unit to control the implant unit.

Various embodiments also include use of a valveless impedance pump implant unit to deliver a variety of drugs and to treat a variety of conditions, examples of which are provided herein.

### BRIEF DESCRIPTION OF THE FIGURES

The following detailed description is better understood when read in conjunction with the accompanying drawings, which are included by way of example, and not by way of limitation, and in which like reference numerals refer to similar elements. In certain cross-sectional and partially cross-sectional views, cross-hatching, stippling and solid black coloring are used to differentiate between separate physical elements, but should not be construed as requiring a particular type of material. Where appropriate, possible material choices for particular elements are provided in the detailed description.

FIG. 1 is a block diagram of an open loop implantable drug delivery sub-system according to some embodiments.

FIG. 2 is a block diagram of an implantable drug delivery sub-system according to additional embodiments.

FIG. 3 is a block diagram of a pump-containing implant unit according to some embodiments.

FIG. 4 is a partially cross-sectional drawing showing passive flow-directing elements which may be incorporated into fluid pathways.

FIG. 5 is a block diagram showing an implant unit integrated circuit according to some embodiments.

FIG. 6 is a block diagram of a circuit configuration for generating actuator drive voltages according to some embodiments.

FIGS. 7 and 8 are schematic diagrams of example oscillator circuits.

FIG. 9 is a schematic diagram of voltage stages in the circuit configuration of FIG. 6.

FIGS. 10 and 11 show waveforms of control signals for switches in voltage stages of FIG. 9.

FIG. 12 is a block diagram of a circuit for generating an actuator drive voltage according to another embodiment.

FIG. 13 is schematic diagram of the drive circuit in FIG. 12.

FIG. 14 is an assembly drawing of a physical configuration for an implant unit according to one embodiment.

FIGS. 15 through 19 are partially cross-sectional drawings showing implant units according to additional embodiments.

FIG. 20 is a partially cross-sectional drawing showing an implant unit according to an additional embodiment.

FIG. 21 is a block diagram of implant units according to additional embodiments.

FIG. 22 is a cross-sectional view of an implant unit according to another embodiment.

FIG. 23 is a cross-sectional view showing use of the implant unit of FIG. 22 with a dual-lumen catheter.

FIGS. 24A through 24D show an implant unit according to additional embodiments.

FIG. 25 shows use of flexible circuit boards in an implant unit and in a patient interface unit.

FIG. 26 is a front view of a handheld patient interface unit according to some embodiments.

FIG. 27 is a block diagram of internal components of the patient interface unit of FIG. 26.

FIG. 28 shows a charging unit according to some embodiments.

FIG. 29 illustrates a headset that incorporates a charging coil.

FIG. 30 is a block diagram of a charging unit according to some embodiments.

FIG. 31 is a block diagram of an implanted drug delivery sub-system that includes components for providing electrical stimulation.

FIG. 32 is a block diagram of an implanted drug delivery sub-system configured to deliver a liquid formulated drug.

### DETAILED DESCRIPTION

Drug delivery systems according to various embodiments use a valveless impedance pump (VI pump) to deliver drug to a desired location in a patient's body. VI pumps can be configured to deliver very low volumes, either intermittently or continuously, over extended periods of time. A VI pump, when incorporated into a unit that is implantable within the patient's body, facilitates a system that can deliver a drug to a specific body region over a prolonged period.

In general, VI pumps employ a pinching element or other type of force-transferring member to mechanically compress a flexible wall of a pump chamber having two openings. The compressions are applied at a location that generally divides the pump chamber into a first sub-chamber located between the first opening and the compression location and a second sub-chamber located between the compression location and the second opening. The first sub-chamber differs from the second sub-chamber (e.g., by having a different volume) such that an applied compression temporarily causes the fluid pressure in one sub-chamber to be greater than the fluid pressure in the other sub-chamber. If the pump chamber walls at the first and second openings are of different material or geometry (or any other factor affecting wave propagation and/or reflection) than the pump chamber wall(s) between those openings, an impedance mismatch, and thus a site for wave reflection, is created. The cumulative effect of constructive pressure wave interaction is to pump fluid in one opening, through the pump chamber, and out the other opening. Controlling the timing, frequency, and displacement of the compression will directly affect the direction and rate of fluid flow. As discussed in more detail below, the fluid chamber can be an elastic tube or can have other shapes, and various types of mechanical actuators can be employed to compress a flexible wall of the chamber. A VI pump is “valveless” in the sense that it does not rely upon valves to generate a net fluid flow, but a VI pump may be part of a fluid path that includes valves for other purposes.

### Definitions

The following definitions apply throughout this specification (including the claims).

Coupled. Coupled components are attached to one another. The attachment can be temporary or permanent and movable or fixed. Coupled components may be attached (temporarily or permanently and movably or fixedly) by one or more intermediate (and not specifically mentioned) components.

Drug. Drug includes any natural or synthetic, organic or inorganic, physiologically or pharmacologically active substance capable of producing a localized or systemic prophylactic and/or therapeutic effect when administered to an animal or human. A drug includes (i) any active drug, (ii) any drug precursor or pro-drug that may be metabolized within an animal or human to produce an active drug, (iii) combinations of drugs, (iv) combinations of drug precursors, (v) combinations of a drug with a drug precursor, (vi) any of the foregoing in combination with a pharmaceutically acceptable carrier, excipient(s), slowly-releasing delivery system, or formulating agent, and (vii) analogs of specific drugs identified herein.

Fluid communication. Two components are in fluid communication if fluid can flow from one component to another. Such flow may be by way of one or more intermediate (and not specifically mentioned) other components. Such flow may or may not be selectively interruptible (e.g., with a valve) or metered.

Target tissue. A region of a patient's body that is to receive treatment from a drug (carried by a vehicle) and/or a region of a patient's body from which the body's own mechanisms will transport that drug to a region that is to receive treatment.

Vehicle. A vehicle is a fluid medium used to obtain drug from one or more masses of solid drug and/or to deliver that drug to a target tissue or to some other desired location. A vehicle may (depending on the vehicle and/or drug being used) obtain drug from one or more solid drug masses through one or more physical mechanisms that include, but are not limited to, any of the following: dissolution of drug from one or more solid drug masses so that the solid drug is a solute within the vehicle, erosion of drug from one or more solid drug masses so that the solid drug is suspended in the vehicle, erosion of drug from one or more solid drug masses and attachment (e.g., adsorption and/or absorption) of such eroded drug to particles (e.g., nanoparticles and/or microparticles) of some other compound that is already suspended in the vehicle, and chemical reaction of drug from one or more solid drug masses with one or more chemical components of a vehicle (or with one or more compounds previously suspended and/or dissolved in the vehicle) to form a new compound that is dissolved and/or suspended in the vehicle. A vehicle can be a bodily fluid, an artificial fluid or a combination of bodily and artificial fluids, and may also contain other materials or drugs in addition to a drug being obtained from one or more solid drug masses. A vehicle may contain such other materials or drugs in solution (e.g., NaCl in saline, a solution of an acid or base in water, etc.) and/or suspension (e.g., nanoparticles and/or microparticles). “Vehicle” also includes a liquid used to carry nanoparticles composed entirely or partially of drug.

### Implantable Drug Delivery Sub-Systems

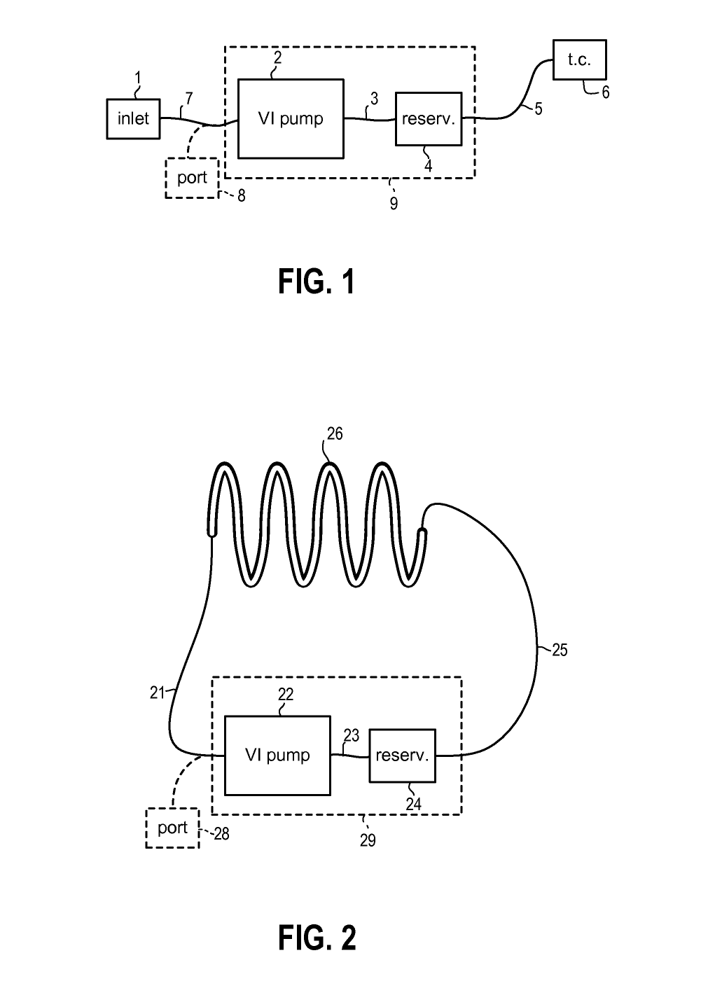

FIGS. 1 and 2 are partially schematic block diagrams showing selected components of drug delivery sub-systems according to certain embodiments. The sub-systems of FIGS. 1 and 2 consist of components that are implanted in the body of a patient. Additional details of the various elements of the implanted subsystem components are discussed below. Each of the sub-systems of the embodiments of FIGS. 1 and 2 is part of a larger system that includes components located external to the patient. These external components are described below under the subheading “System Components External to the Patient.”

FIG. 1 is a block diagram of an open loop implantable drug delivery sub-system according to some embodiments. The sub-system of FIG. 1 includes an inlet 1 for receiving a vehicle, a valveless impedance (VI) pump 2, a solid drug reservoir 4, and a terminal component 6. In some embodiments, VI pump 2 is contained within an implant unit that also contains control electronics, a battery and other elements described below. Reservoir 4 is a separate implant unit that is coupled to (and in fluid communication with) the VI pump implant unit via catheter 3 or another fluid path. In other embodiments, and as indicated by the broken line 9 around blocks 2 and 4 in FIG. 1, VI pump 2 and reservoir 4 are contained in a single implant unit. Reservoir 4 is in fluid communication with terminal component 6 via catheter 5 and VI pump 2 is in fluid communication with inlet 1 via catheter 7. In operation, VI pump 2 draws the vehicle from inlet 1 and propels that vehicle through reservoir 4 out of terminal component 6. Terminal component 6 is implanted in or near a target tissue. Vehicle passing through reservoir 4 obtains solid drug from one or more masses of solid drug held within reservoir 4, with the vehicle and drug then delivered to the target tissue through terminal component 6. In some embodiments, catheters 7 and/or 5 might also be omitted (e.g., inlet 1 may be an opening in a housing of VI pump 2).

Depending on the specific embodiment and use thereof, inlet 1 may have a variety of configurations and receive a vehicle from a variety of sources. In some embodiments, the vehicle is a physiological fluid collected from within the patient's body (e.g. interstitial fluid, perilymph, vitreous, or cerebrospinal fluid). In such embodiments and uses, inlet 1 may be the open end of catheter tube 7, with the other end of tube 7 connected to an inlet opening of a housing for VI pump 2. Inlet 1 is placed in a region of a patient's body from which a vehicle can be drawn (e.g., the ear, the brain, the spine, the eye or an interstitial space). Inlet 1 in some embodiments includes a porous membrane or three-dimensional porous filter to prevent particles from clogging the system. In still other embodiments, inlet 1 may be a trans- or subcutaneously implanted refillable septum-top reservoir containing a supply of vehicle (e.g., Ringer's solution, Ringer's lactate, saline, physiological saline, or artificial perilymph). Examples of trans- and subcutaneously implantable ports that can serve as vehicle reservoirs are described in the following commonly-owned U.S. patent application Ser. Nos. 11/337,815 (published as Pub. No. 20060264897), Ser. No. 11/414,543 (published as Pub. No. 20070255237), and Ser. No. 11/759,387 (published as Pub. No. 20070287984). Other types of implantable reservoirs can be used, however.

In some embodiments, an implanted port is used as a liquid reservoir for holding and supplying vehicle, with the supply of vehicle in the port/reservoir replenished by injection of additional vehicle through the patient's skin and the elastic septum of the port. As an alternative embodiment, an implanted port may be in fluid communication with a separate liquid reservoir which contains a bellows and a metering orifice. The bellows would allow the injected vehicle to accumulate within the reservoir and then the metering orifice would release the vehicle into the pumping mechanism at a slower rate. In still other embodiments, and as shown by the broken line port 8 in FIG. 1, an implanted port may be used to inject an additional drug (e.g., a liquid-formulated drug to be delivered in combination with a drug obtained from solid drug reservoir 4) into a flow of vehicle from a location in the patient's body (e.g., a source of a bodily fluid vehicle in which inlet 1 is implanted) to VI pump 2. Port 8 could also be included in the same housing with VI pump 2 and/or solid drug reservoir 4.

Reservoir 4 contains a supply of drug in solid form. That drug may be a single mass or multiple masses (e.g., pellets). The drug may be a single drug, a combination of drugs, or a combination of one or more drugs with other materials (e.g., a binder or a degradable release system). The drug contained in reservoir 4 may also be a mass of nanoparticles and/or microparticles. As indicated above, reservoir 4 may be a separate implant unit and have its own housing, or it may be contained with VI pump 2 in a common (or coupled) housing as part of a combination implant unit. Reservoir 4 may include screens for preventing migration of solid drug and/or hold the drug mass(es) in a cage-like enclosure. Reservoir 4 may further contain an antibacterial filtration system. An antibacterial filtration system can alternatively be included as a separate component in fluid communication with drug reservoir 4. Examples of solid drug reservoirs that can be employed in at least some embodiments are described in the previously identified Ser. No. 11/414,543 and Ser. No. 11/759,387 applications, as well as in commonly-owned U.S. patent application Ser. No. 11/780,853 (published as Pub. No. 20080152694). Although reservoir 4 is shown downstream of VI pump 2 in FIG. 1, reservoir 4 may be located upstream (i.e., on the inlet side) of VI pump 2 in other embodiments.

Terminal component 6 will vary based on the manner in which the system of FIG. 1 is to be used. In some implementations, terminal component 6 may be a simple open end of catheter 5. When delivering drugs to the inner ear, terminal component 6 may be a needle which is sized and configured for easy and effective movement within the middle ear for performing round window injections or injections through the cochlear bone. Such a needle may be straight, or it may have one or more bends or curves designed for round window injection or insertion through a hole in the cochlear bone and/or the promontory bone and/or the temporal bone. Alternatively, a needle with a blunt tip may be inserted through a hole drilled in the bone wall of the basal turn for access to the scala tympani, with the bone needle forming a leak-proof passage through the bone (i.e., only allowing fluid to pass via the needle interior). In such an embodiment the needle may include an insertion stop which could be formed from a porous biocompatible material such as titanium, titanium alloys, stainless steel, etc. Porous or non-porous titanium may be coated with ceramic such as hydroxyapatite or plastic, or treated with chemicals and/or heat (e.g., NaOH treatment and heat treatment), to help hydroxyapatite forming during bone tissue integration. When placed into a specially prepared pocket within the bone, the bone may then grow into and over the insertion stop to form a permanent connection. Examples of terminal components for delivery of drugs to the inner ear are described in commonly-owned application Ser. No. 11/337,815.

For ophthalmic delivery of drugs, terminal component 6 may be a soft tissue cannula (e.g. a small-diameter flexible polymeric tube made from, e.g., polyimide, a fluoropolymer, silicone, polyurethane or PVC) or a rigid needle which passes through an incision in the sclera and injects fluid into specific regions within the inner eye. Depth and location of insertion of a terminal component depends on which region is being targeted in the eye. The cannula or needle may have an insertion stop which controls the depth of insertion. One preferred location for the incision is in the pars plana. Other preferred locations for terminating the cannula for drug delivery may be in the vitreous or the anterior chamber, allowing drugs to be delivered in controlled doses to the precise area of the eye. The terminal end of the catheter may be fixed, for example via suture, surgical tack, a tissue adhesive, or a combination thereof, to tissue near the outer surface of the eye. When attached, the catheter does not affect or otherwise restrict movement of the eye. Examples of devices and methods for ophthalmic drug delivery are disclosed in commonly-owned application Ser. No. 11/780,853.

FIG. 2 is a block diagram of an implantable drug delivery sub-system according to additional embodiments. The sub-system of FIG. 2 includes a VI pump 22, a solid drug reservoir 24, and a fluid exchange element 26. Unlike embodiments corresponding to FIG. 1, the system of FIG. 2 circulates a vehicle in a closed loop. In particular, VI pump 22 propels a vehicle through reservoir 24. The vehicle then flows to an inlet of exchange element 26. Element 26, which is implanted in a target tissue, is formed from a material that allows drug in the vehicle to pass through and be delivered to the target tissue. Drug depleted vehicle then flows from an outlet of element 26 and returns to an inlet of VI pump 22 via catheter 21.

VI pump 22 and reservoir 24, which are similar to VI pump 2 and reservoir 4 of FIG. 1, are in some embodiments separate implant units and placed into fluid communication using a catheter 23. In alternate embodiments VI pump 22 and reservoir 24 may share a common housing as part of a combination implant unit (represented by broken line 29). Exchange element 26, which is in at least some embodiments placed into fluid communication with reservoir 24 and VI pump 22 through tubing 25 and 21, can be formed from a variety of materials. In certain embodiments, element 26 is a tube formed from a semi-permeable membrane or hollow fiber and includes multiple loops or coils that increase the amount of surface area available for migration of drug from a vehicle (flowing in element 26) to a bodily fluid in the target tissue where element 26 has been implanted. The length of element 26 can thus be selected so as to control (at least in part) the dosage of delivered drug. Although reservoir 24 is shown downstream of VI pump 22 in FIG. 2, reservoir 24 may be located upstream (i.e., on the inlet side) of VI pump 22 in other embodiments.

In other embodiments, element 26 may be formed from multiple tubes. For example, the inlet of element 26 can branch into multiple tubing sections through which vehicle can flow in parallel, with those sections rejoining at tube 21 for return of vehicle to VI pump 22. In still other embodiments, exchange element 26 is not tubular, and is instead formed from two flat pieces of semipermeable membrane (or one piece folded over on itself) that are sealed along the edges; vehicle with drug is input into one end (e.g., through a tube inserted and sealed into a first edge) and drug-depleted vehicle flows from another end (e.g., through a separate tube inserted and sealed into a second edge). As with tubular embodiments, the length of a flat (or flattened) exchange element can be varied to control drug dosage.

In still other embodiments, and as shown by the broken line port 28 in FIG. 2, an implanted port may be added to the sub-system of FIG. 2. Port 28 can then be used to inject an additional drug (e.g., a liquid-formulated drug to be delivered in combination with a drug obtained from solid drug reservoir 24) into the vehicle circulating within the closed loop of the implanted sub-system. Port 28 could also be used to replenish any small amounts of vehicle that might escape from the closed loop sub-system after implantation for an extended period. Port 28 could also be included in the same housing with VI pump 22 and/or solid drug reservoir 24.

### VI Pump Implant Units

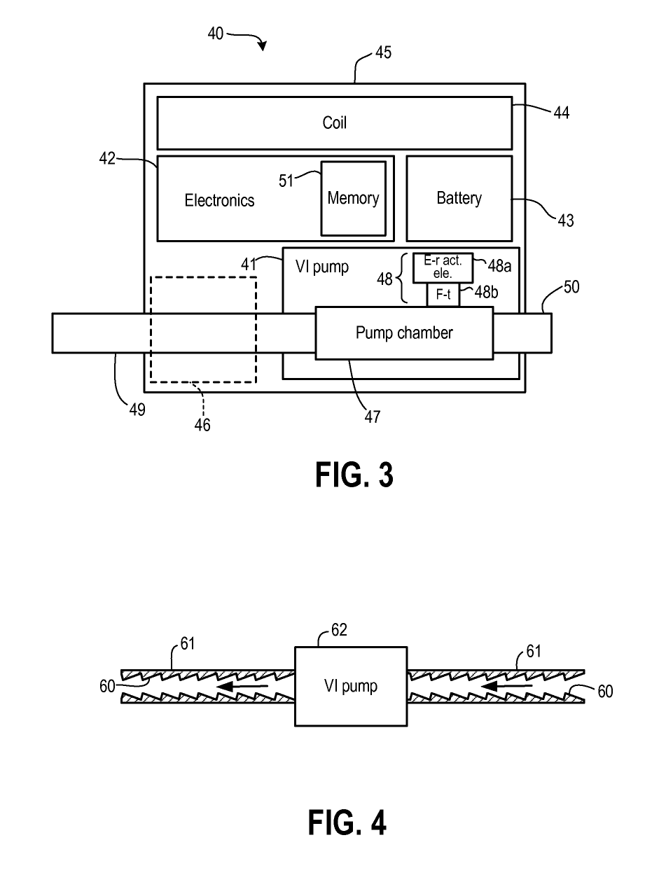

A pump-containing implant unit according to certain embodiments includes multiple components to form an operable drug delivery sub-system. As seen in the block diagram of FIG. 3, a pump-containing implant unit 40 according to certain embodiments may include a VI pump 41, control electronics 42, a battery 43, a communication/charging coil 44, and a housing 45. In some embodiments, the pump implant also includes a drug reservoir 46 (shown in broken lines), while in other embodiments a drug reservoir may be a separately implanted physical component. As in other embodiments, drug reservoir 46 may be located up- or downstream of pump 41.

VI pump 41 includes a compressible pump chamber 47. An actuator 48 comprises an electro-reactive actuating element 48a and a force-transferring member 48b (in contact with a chamber wall) and is configured to compress chamber 47. In some cases, for example, electro-reactive actuating element 48a may be a piezoelectric element that exerts force in response to an applied drive voltage, and force-transferring member 48b may be a rod, arm or other member (or collection of members) coupled to the wall of pump chamber 47 at a compression location. As another example, force-transferring member 48b may be a permanent magnet or other magnetically-reactive material that is coupled to the pump chamber wall at the compression location, and electro-reactive actuating element 48a may be an electromagnet. In still other embodiments, electro-reactive actuating element 48a and force-transferring member 48b may be combined (e.g., a piezoelectric element directly contacting the pump chamber wall). As previously indicated and as described in more detail below, chamber 47 may be a tube. As but one example, a 14.8 mm length of silicone tube (having a 0.30 mm inner diameter and a 0.64 mm outer diameter), attached at the ends to 25 gage stainless steel tubes, will deliver an 86 nanoliter (n1) bolus dose of water when compressed (at 40 Hz for 3 cycles) at a position located 3.8 mm from one of the stainless steel tube/silicone tubing connections. In other embodiments, operating frequency may range from 1 to 5000 Hz.

Pump chamber 47 need not be tubular. For example, pump chamber 47 could be a flexible fluid pathway having an oval, polygonal or other non-circular cross-section. As used herein, “tube” and “tubing” include fluid conduits having non-circular cross-sections.

In at least some embodiments, pump 41 may be able to operate intermittently for a period of 3 to 5 years (and perhaps as much as 20 years) without degradation of the pump chamber.

In some embodiments, and as also discussed below, pump 41 may utilize a pump chamber that includes one or more thin flexible membranes. The membrane may be coupled to rigid surrounding material and vibrated by a magnetic or piezoelectric actuator, which actuator may be laminated to the membrane surface. In one embodiment, the actuator vibrates the membrane at an asymmetric location along the length of the membrane covering the fluid filled cavity of the pump chamber and creates a flow impedance mismatch between the membrane and the rigid cavity ends constraining the membrane. The actuator is centered on the membrane in other embodiments, with the membrane located on an asymmetric location with respect to the chamber fluid cavity. In some such embodiments, a flow impedance mismatch can be created by the pump chamber cavity having greater cross-sectional area than the inlet and outlet cavities. In certain embodiments, an additional flow-directing flow impedance mismatch may be created between one end of the cavity (near a first opening and having a larger cross-sectional area) and another end of the cavity (near a second opening and having a smaller cross-sectional area). A membrane VI pump can be built in layers using silicon or glass, where the fluid cavity is either machined or etched. The membrane may be made of a flexible biocompatible and drug compatible material such as PDMS, silicone, fluoropolymer or polyurethane and having a thickness of, e.g., less than 0.005 inches.

In some embodiments a hydrophobic vent is incorporated into the inlet side of VI pump 41 to evacuate entrained air which may negatively affect pump operation if introduced into the compressed section of pump chamber 47. The vent may be a hydrophobic membrane incorporated into the inlet tubing, or the inlet tubing itself may be made of a hydrophobic porous material. As an example, the membrane or tubing may be made of porous PTFE with a pore size of 0.02 micrometers. In some embodiments air elimination component(s) may be made of a hollow fiber or of a porous plastic, metal, ceramic or composite.

In some embodiments VI pump 41 includes rigid tubing connectors 49 and 50, with each connector being laser-welded or otherwise sealed to the housing 45 of implant unit 40 at one end and being attached to pump chamber 47 at the other end. The interfaces between rigid tubes 49 and 50 and pump chamber 47 provide locations for pressure wave reflection when chamber 47 is compressed and also provide attachment points for catheters providing fluid communication to other implanted components. Ends of the rigid tubes 49 and 50 may include barbs and/or may have rings to tightly clamp chamber 47 and catheters (not shown) to tubes 49 and 50. The rings may incorporate a feature that forces chamber 47 to match the inner diameter of the rigid tubes so that there is no place for an air bubble to stop.

In other embodiments, rather than having two rigid connector tubes, the entire fluid pathway of the VI pump may be a single tube with a flexible section for actuation. This can be manufactured by inserting rigid tubes into a flexible tube to form dual-layered tubing with a small section of single-layered flexible tubing. The impedance mismatch in this embodiment is derived from difference in hardness and diameter between the inner and outer tubes. As an example the rigid tubes may be made of PTFE, and the flexible outer tube may be made of silicone. Silicone tubing may be swelled with heptane to allow for initial insertion of the rigid tubing when manufacturing the dual-layered tubing. The tubing may be attached to the housing (at the inlet and outlet locations) with a medical grade adhesive such as silicone adhesive, UV curing epoxy, or other adhesives. Another method of making the single diameter tube is to bond rigid tubing to the ends of the flexible tubing so that the inner and possibly also the outer diameters are constant, but the flexibility varies.

In one embodiment the entire fluid pathway of pump 41 may be a single flexible tube. Rigid rings may be fastened to the outside of the flexible tube to provide locations for pressure wave reflection, and to provide locations for supporting the tube within the pump housing. The rigid tubes can be bonded or fastened (via laser welding, as an example) to the pump housing.

In some embodiments, a VI pump is in fluid communication with tubing that has different flow resistances in the forward and reverse directions so as to increase system resistance to backflow and enhance the reliability of a one-way delivery system. FIG. 4 illustrates passive flow-directing elements which may be incorporated into fluid pathways to facilitate flow in one direction. This configuration avoids wear and fatigue associated with check valves and reduces the risk of check valve clogging. In the embodiment of FIG. 4, the internal surfaces 60 of tubing 61 leading to and/or from a VI pump 62 may have barbed or scaled features that allow fluid to flow more easily in one direction (represented by arrows). In another embodiment, the fluid pathway may include looping channels similar to those disclosed in U.S. Pat. No. 5,876,187.

Returning to FIG. 3, electronics 42 includes logic and circuits to control the time and duration of compressions applied to chamber 47 by actuator 48. In some embodiments, the frequency and amplitude of compressions is also controlled. In some embodiments, electronics 42 also include circuits providing a manual on/off control for VI pump 41, which on/off may be toggled by an accelerometer switch in implant unit 40 that is triggered by tapping on the patient skin (near implant unit 40) in a predetermined pattern. Electronics 42 also includes oscillator and clock circuits used to control pump operation and other functions within implant unit 40. As discussed in more detail below, electronics 42 may also include circuits for generating voltage levels needed to drive actuator 48.

Electronics 42 further includes control circuits and logic that control the rate, timing and end condition of charging of battery 43. The battery control circuits and logic also monitor various parameters for battery 43 such as charging and discharging current and voltage, supply voltage, stop charge current and voltage, and temperature. The battery control circuits and logic also store charge history and/or other data regarding battery 43 in memory 51.

Electronics 42 also include communication circuits and logic that transmit data from implant unit 40 to an external device (e.g., a patient interface unit as described below) and that receive instructions from an external device. The communications circuits and logic also identify external communications permitted to interface with implant unit 40 (using, e.g., a password) and perform error recognition and correction on received communications.

Electronics 42 further includes memory 51 having both volatile and non-volatile memory components. In addition to battery data, memory 51 can be used to store instructions controlling operation of pump 41. For example, firmware in electronics 42 may access data stored in memory 51 that corresponds to one or more dosing sequences by which pump 41 should be activated. The dosing sequence data may include times at which actuator 48 is to be activated or deactivated, a duty cycle for actuator 48 (i.e., how many compressions should be applied or how long electro-reactive actuating element 48a should be energized), amplitude of compressions to be applied by actuator 48, etc. Dosing sequence data, limits of dosing (e.g., maximum dosage and/or minimum time between patient-initiated dosing cycles, etc.) and other parameters of implant unit 40 operation can be stored in memory 51 in response to communications from an external device (e.g., a patient interface unit and/or a charging unit). Memory 51 may also store communication software and/or other control software, which software may also be updatable or otherwise modifiable in response to communications from an external device.

Coil 44 is used to communicate with an external device and to charge battery 43. Coil 44 complies with ISO 60601 requirements for electromagnetic safety and is configured to operate in a frequency range that is established for medical devices. When a fluctuating magnetic field (generated from an external device in close proximity to the patient) is present, coil 44 will generate an AC voltage and current. A voltage converter in electronics 42 will rectify the AC voltage and current and transform it into a form required by other elements of implant unit 40. The power output from coil 44 can be used to charge battery 43, for communication, etc. In some embodiments, the electro-reactive actuating element for VI pump 41 is not powered by a battery, and an external magnetic field may be cycled on and off to cause pumping action. In some applications an external magnetic field will be on continuously and the pump will run until the field is disengaged.

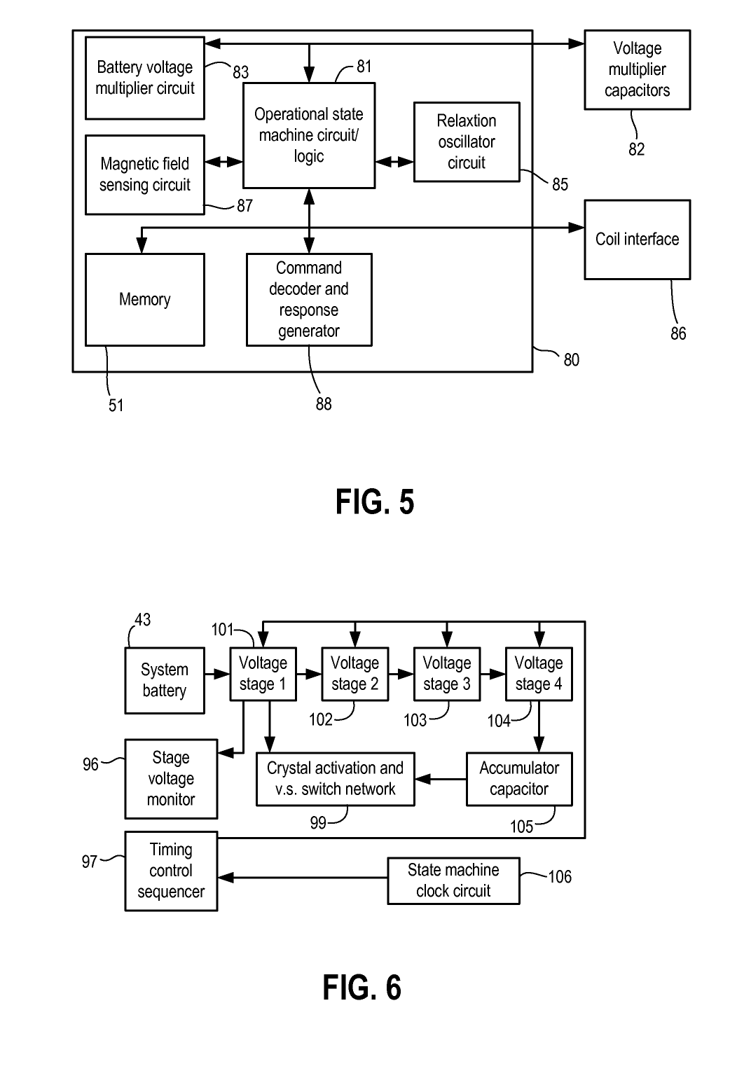

In some embodiments, much of electronics 42 can be contained on a single high voltage integrated circuit (IC). FIG. 5 is a block diagram showing an IC 80 according to some embodiments. State machine circuitry 81 controls the operational mode of implant unit 40. Separate sequences can be executed for various functions (electro-reactive actuating element control, battery charging, communication, etc.) and cycled as necessary to extend battery life. In some embodiments, and as described below, state machine circuitry 81 also includes switches for controlling connections to voltage multiplier capacitors 82 that may be located external to IC 80. State machine circuitry 81 also creates separate sequencing clock signals for battery voltage multiplier circuit 83. In an active mode, state machine 81 will cause multiplier circuit 83 and external capacitors 82 to generate the voltage needed to drive actuator 48 and will control switching of that drive voltage to actuator 48. State machine circuitry 81 also monitors battery 43 voltage and controls a shutdown circuit for a charging coil 44 to prevent overcharging. Relaxation oscillator circuit 85 provides a system clock for state machine 81. In some embodiments, oscillator 85 is also the source of clock signals for voltage multiplier circuit 83 and the source of a further divided clock signal controlling activation frequency of actuator 48. Coil interface 86, which is in some embodiments not located on IC 80, is a passive circuit that rectifies a signal from coil 44. Coil interface 86 also includes a resonate circuit shutdown and an over voltage sensing circuit. The over voltage detector reduces the Q (Quality factor, a ratio of center frequency to bandwidth, which is also a ratio of energy storage to energy absorbed) of the resonate circuit if a received voltage would potentially damage IC 80. The over voltage detector can also include a threshold detector that sends an interrupt to state machine 81 if a signal of sufficient magnitude is detected. This interrupt can activate state machine 81 if implant unit 40 was in a shutdown or standby mode and cause state machine 81 to transition into a communication and charging mode. Battery voltage multiplier circuit 83 maintains a supply voltage for actuator 48. When state machine 81 is in an active mode, power supply to actuator 48 is monitored and switching of capacitors 82 is initiated if that power supply drops below a threshold value.

Magnetic field sensing circuit 87 detects the presence of a magnetic field from an external device. Command decoder and response generator 88 includes circuits and logic for, e.g., decoding communications, executing commands, generating communications (e.g., for export of data stored in memory 51), storing data to memory 51, etc.

In some embodiments, electro-reactive actuating element 48a (FIG. 3) is piezoelectric and requires a drive voltage that is significantly higher than that of battery 43. FIG. 6 is a block diagram of a circuit configuration for generating such voltages according to some embodiments. As will be apparent in view of the following, the block diagram of FIG. 6 encompasses components that may be contained within blocks 81 and 83 of IC 80 (FIG. 5) and charge capacitors 82 external to IC 80. A circuit configuration according to FIG. 6 produces a fixed voltage of 2^{N}×B, where N is the number of voltage stages and B is the voltage of battery 43. Although this equation ignores resistive drops across switch networks, this is a reasonable assumption, as the total current flow in the charging system is negligible. The configuration of FIG. 6 includes 4 stages to give 16× battery voltage, but the total voltage can be scaled by removing or adding one or more stages.

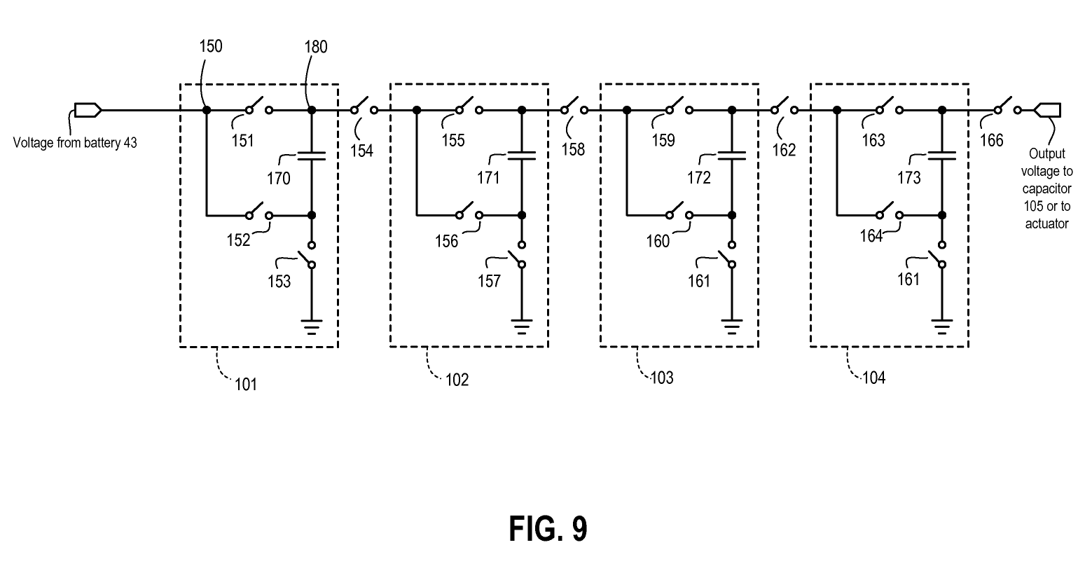

A typical operational voltage available from rechargeable batteries is approximately 3 volts, which value is assumed in the following description. Integrated circuit technologies that support higher voltages typically will allow up to 50 volts. The output of the first voltage stage 101 is 2×B (6 volts). The output of the second stage 102 is 12 volts, the output of third stage 103 is 24 volts, and the output of fourth stage 104 is 48 volts. The output of the fourth stage 104 is stored in an accumulator capacitor 105 as a constant supply voltage. As described more fully below in conjunction with FIG. 9, each of voltage stages 101, 102, 103 and 104 includes a capacitor and a switch network. Higher capacitance values are used in higher voltage stages. All of the switches for the voltage stages are incorporated into IC 80. The switching rate of capacitors in voltage stages 101, 102, 103 and 104 will be high relative to the rate of actuator 48 movement. If a stable voltage applied to a piezoelectric crystal of electro-reactive actuating element 48a is desired, accumulator capacitor 105 may be included. In some applications, a high frequency variation in applied crystal voltage may not be a detriment, and the capacitor of fourth stage 104 can be used as the final output so as to eliminate accumulator capacitor 105.

A piezoelectric crystal of electro-reactive actuating element 48a can be modeled as a series and parallel resonant circuit. In general, the series and parallel resonant frequencies of that circuit model will be far above those used for any reasonable mechanical actuation needed for pump 41. Accordingly, the crystal of electro-reactive actuating element 48a can be modeled as a pure capacitance. The process of charging and discharging the capacitance of the electro-reactive actuating element 48a crystal will cause flexing and relaxing, respectively. During the flex phase, the electro-reactive actuating element 48a crystal stores energy from accumulator capacitor 105 (or from the last voltage stage if accumulator capacitor 105 is omitted). During the relax phase, the energy stored in the electro-reactive actuating element 48a crystal is returned to the voltage stages in sequence. When the electro-reactive actuating element 48a crystal switches from flex to relax, the voltage is initially higher than the voltage on the capacitor of third stage 103. Under control of a stage voltage monitor circuit 96, the crystal is first discharged into third stage 103. The reduced voltage on the electro-reactive actuating element crystal is then discharged into second stage 102, and finally into first stage 101. This process allows the recovery of the energy stored in the electro-reactive actuating element and reduces the energy required from battery 43.

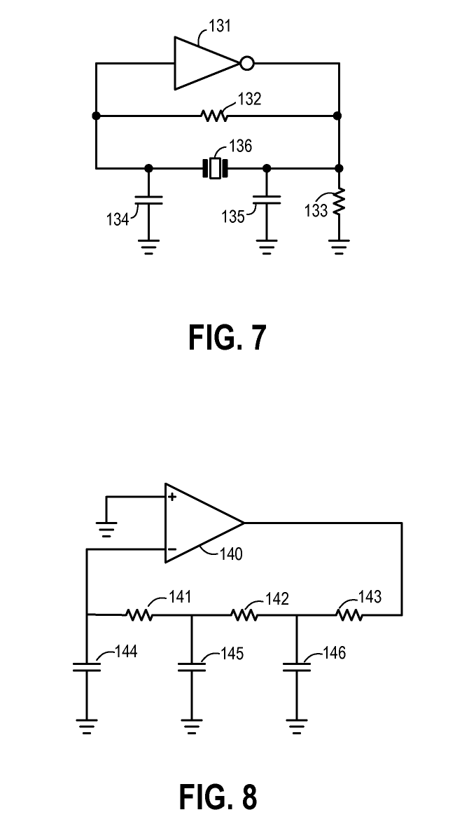

State machine clock circuit 106, which may be part of the state machine circuit 81 of IC 80 (FIG. 5), may be the system oscillator for IC 80 or may be a separate oscillator dedicated to run the voltage converter portion of the circuit of FIG. 6. Various other types of oscillator circuits could be used. A typical oscillator circuit that can be used is shown in FIG. 7, and includes an inverter 131, resistors 132 and 133, capacitors 134 and 135 and crystal 136. Because the system oscillator for IC 80 may operate at a higher frequency than is required for voltage conversion operations, state machine clock circuit 81 may further include a divider to create a reduced-frequency version of a clock signal from the system oscillator. In some embodiments, the output frequency of state machine clock circuit 81 may be adjustable to affect the system performance.

If there is no system oscillator for other electronic components of an implant unit, or if incorporating a system oscillator into a circuit for generating the accumulator 48 drive voltage is undesirable, state machine clock 106 may include an independent oscillator. In some such embodiments, a simple RC oscillator such as the one shown in FIG. 8 could be used. The oscillator circuit of FIG. 8 includes an operational amplifier (op amp) 140, resistors 141, 142 and 143, and capacitors 144, 145 and 146. Many other configurations would also be acceptable.

FIG. 9 is a schematic diagram of voltage stages 101, 102, 103 and 104. Each of switches 151 through 166 could each be implemented as a MOSFET transistor on IC 80 (as part of crystal activation and v.s. switch network 99) able to handle the voltages expected at the stage in which the switch is located. Capacitors 170, 171, 172 and 173 are discrete components external to IC 80.

Each voltage multiplier stage executes a 2 step cycle. Focusing on first stage 101, for example, switches 151 and 153 are closed and switches 152 and 154 are open on the first half of the cycle. During this time, the voltage input from battery 43 at node 150 charges the capacitor 170. During the second half of the cycle, switches 152 and 154 are closed and switches 151 and 153 are open. In this half of the cycle, the voltage output at node 180 is twice the voltage input, and is made available to second stage 102. Second, third and fourth stages 102, 103 and 104 operate in a similar manner, except that the frequency for each successive stage is half of the frequency of the previous stage. In other words, the frequency of the switching cycle for second stage 102 is half that of first stage 101, the frequency of third stage 103 is half that of second stage 102, etc. The timing of the switches in voltage stages 101, 102, 103 and 104 is under the control of timing control sequencer circuit 97.

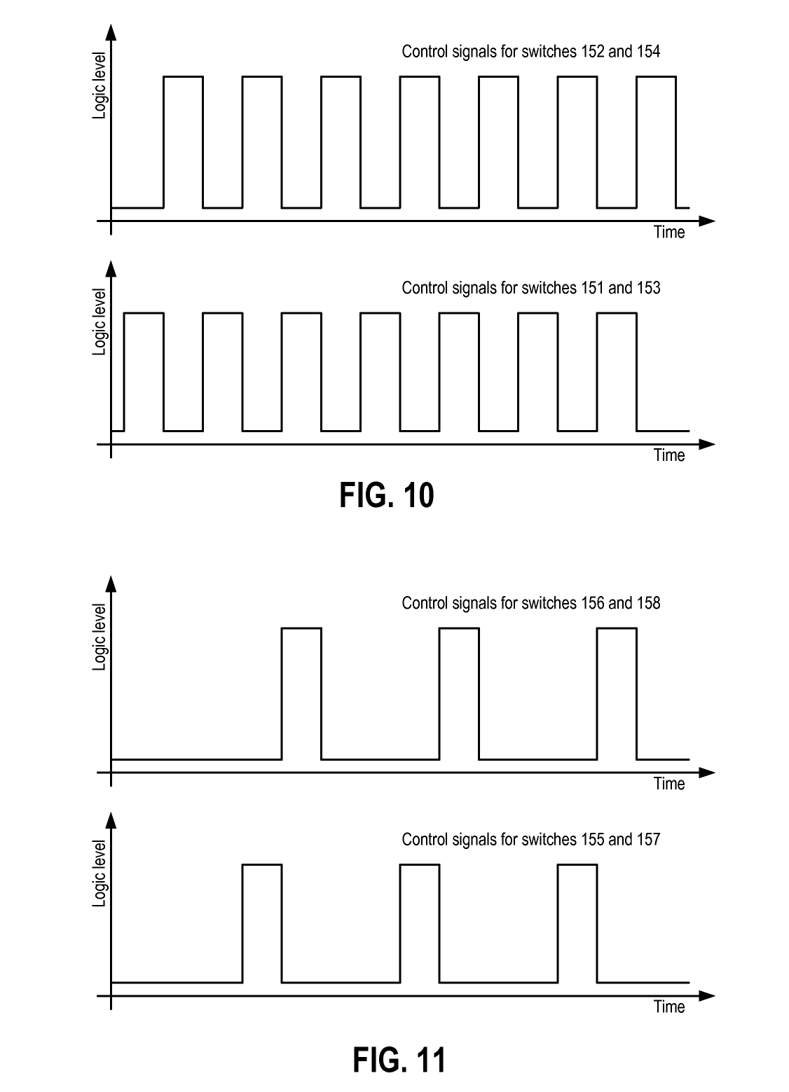

FIG. 10 shows 2 time-based waveforms (logic level voltage on the vertical axis versus time on the horizontal axis) illustrating the control signals for switches 151, 152, 153 and 154 in first stage 101. A high logic level voltage is assumed to cause a switch to close. The lower graph in FIG. 10 shows the control signal for switches 151 and 153 and the upper graph shows the control signal for switches 152 and 154, with the upper and lower graphs having the same time axis. As seen in FIG. 11, two time-based waveforms (logic level voltage on the vertical axis versus time on the horizontal axis) illustrating the control signals for switches 155, 156, 157 and 158 in second stage 102, second stage 102 runs at half the frequency of first stage 101. This allows the first stage capacitor 170 to recharge for a full cycle of the first stage between transitions of the second stage. The lower signal graph of FIG. 11 shows the control signal for the charging pair of switches (155 and 157) and the upper graph of FIG. 11 shows the control signal for the discharging pair (156 and 158). The upper and lower graphs of FIG. 11 have the same time axis as the upper and lower graphs of FIG. 10.

As previously indicated, the sub-circuit of FIG. 9 is scalable. Higher voltages are achieved by increasing the number of stages and lower voltage can be produced with fewer stages. The circuit stage inputs are connected, with outputs of lower stages connected to inputs of higher stages.

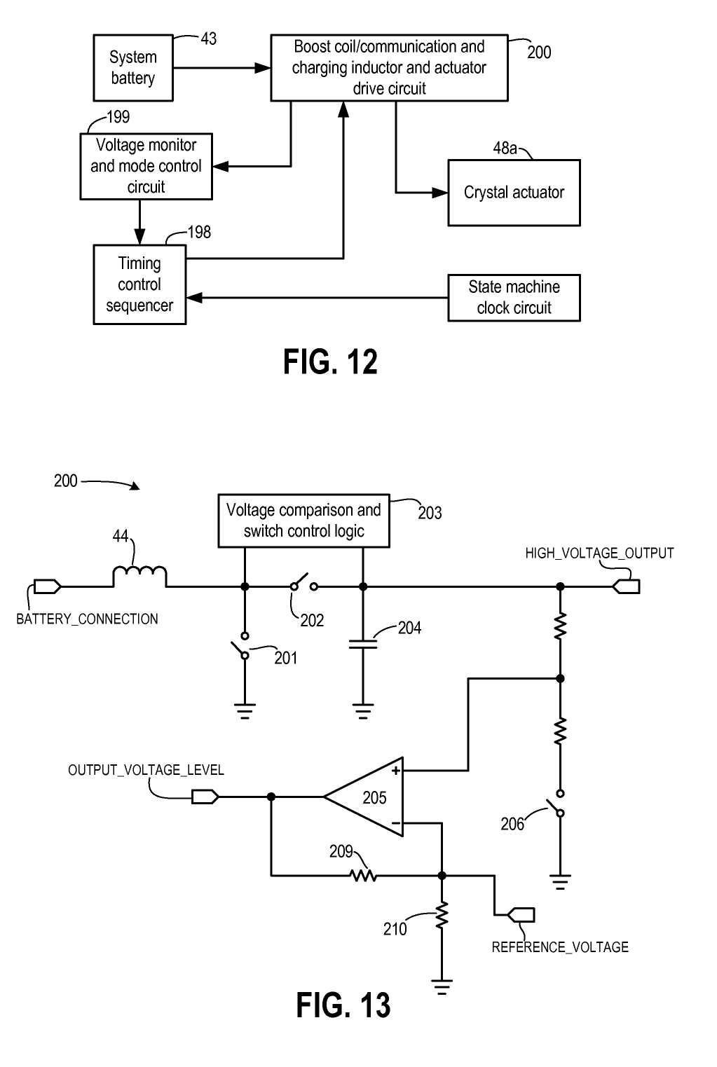

FIG. 12 is a block diagram of a circuit configuration for generating a drive voltage for piezoelectric electro-reactive actuating element 48a (FIG. 3) according to another embodiment. The voltage generating circuit of FIG. 12 is a constant duty cycle switched sampling boost converter that employs charging/communication coil 44 as the inductor of the boost converter circuit. FIG. 13 is schematic diagram of the drive circuit 200 in FIG. 12. When not in charge mode, switch 201 remains open and no current flows from battery 43. During the charging cycle, switch 201 closes temporarily. The battery 43 voltage is applied across communication and charging coil 44. Current through coil 44 increases and the magnetic field builds up around the windings and stores energy from battery 43.

In a traditional boost converter circuit, switch 202 and the voltage comparison and switch control logic sub-circuit 203 is replaced with a diode. Switch 202 and the voltage comparison and switch control logic sub-circuit 203 perform a similar function, but without power losses associated with a diode voltage drop. Whenever the voltage comparison and switch control logic sub-circuit 203 detects a voltage on the coil 44 side of switch 202 that is greater than or equal to the voltage on the capacitor 204 side, switch 202 is closed. When switch 201 opens, energy stored in coil 44 causes the voltage on the right side of coil 44 to rise. When switch 202 closes, current generated by the magnetic field of coil 44 charges capacitor 204.

The amount of energy transferred to capacitor 204 depends on the amount of energy stored in coil 44, which is in turn proportional to the time that switch 201 is closed and to the time that power is supplied to the operational amplifier (205) of the comparator sub-circuit (described below). Traditional boost converter circuits vary the duty cycle to regulate the output voltage. This may be necessary for a system operating under varying load conditions. As the load on the circuit of FIG. 13 is generally fixed, however, inefficiencies associated with a varying duty cycle can be eliminated.

Periodically, switch 206 closes and power is applied to comparator amplifier 205. During this sampling time a fraction of the voltage on capacitor 204 is compared against a Reference_Voltage adjusted by the hysteresis offset created by resistors 209 and 210. If the output voltage (High_Voltage_Output) is below a threshold set by Reference_Voltage, the Output_Voltage_Level signal goes high, which then increases the voltage at the non-inverting input of op amp 205. Accordingly, Output_Voltage_Level remains high notwithstanding minor fluctuations in 204 voltage. A high Output_Voltage_Level is noted by the voltage monitor and mode control circuit 199 (FIG. 12), which then puts the boost converter of FIG. 13 into charge mode.

If the fraction of the High_Voltage_Output level reaching the inverting input of op amp 205 is greater than the Reference_Voltage, the comparator produces a low signal at the output of op amp 205, resistors 209 and 210 reduce the amount of Reference_Voltage reaching the non-inverting input of op amp 205, and the Output_Voltage_Level signal remains low. A low Output_Voltage_Level signal is noted by the voltage monitor and mode control circuit 199, which then puts the boost converter of FIG. 13 into standby mode. The sampling of the High_Voltage_Output signal is momentary with a very low duty cycle.

The voltage monitor and mode control circuit 199 periodically samples the output voltage as described above and stores the result for the timing control sequencer circuit 198. The timing control sequencer circuit 198 monitors the mode control signal. When in charge mode, timing control sequencer 198 pulses switch 201 to charge coil 44 and then transfer the energy to capacitor 204. When in standby mode, switch 201 remains open. Timing control sequencer 198 also controls the voltage applied to the crystal of electro-reactive actuating element 48a. During the flex portion of the electro-reactive actuating element 48a signal, the High_Voltage_Output is applied across the crystal. During the relax portion of the cycle, only the battery voltage is applied across the crystal of electro-reactive actuating element 48a.

The boost circuitry of FIG. 13 can be operated at a fixed duty cycle chosen to match the impedance of battery 43 and to optimize efficiency. Output voltage sampling is only performed periodically, and for very brief periods of time. Output voltage sampling frequency can also be programmable so as to accommodate a variety of load situations. The boost converter switching is performed at a high speed to minimize heating and switch losses. The boost circuit charges a capacitor to a desired voltage and is shutoff until the load reduces the voltage below a preset minimum. A hysteresis can be set so that a single cycle of the boost circuit will result in full recharge.

Returning to FIG. 3, a single coil 44 within implant unit 40 can be used for charging battery 43, communicating with an external handheld control unit, and controlling shutdown of pump 41. Coil 44 can be connected in parallel with a tuning capacitor (not shown) and be sensitive to a narrow band of frequencies in the 110 KHz to 130 KHz band. When a magnetic field within the bandwidth of the tuned circuit is sensed, electronics 42 of implant unit 40 will go to communication and charging mode.

Uplink communications from a patient interface unit (PIU) to implant unit 40 may be formatted to include an 8 bit identification code (that may be related to an identifier for a specific implant unit 40), followed by a 4 bit command. The data may be FSK encoded and include 4 bits of error identification. The data stream may be transmitted 3 times in a 10 mS burst so as to prevent crosstalk from an external PIU communicating with two implant units located within patients who are in the same room. Examples of communications that may be sent to implant unit 40 include commands from a PIU or other external device to modify pump parameters. Software within implant unit 40 (e.g., firmware within electronics 42 and/or code stored in memory 51) controls variable parameters such as dosing frequency and dosing amount corresponding to one or more dosing sequences. Communications may be in a frequency range established for medical devices and configured such that implant unit 40 is able to respond to a communication in less than one minute.

Downlink communications from implant unit 40 to a PIU can be effected by momentarily shorting charging coil 44. A short on coil 44 will cause a higher rate of current in a nearby PIU coil and can be detected. The downlink data may contain responses to uplink commands, e.g., an “acknowledge” or data from the requested register in memory 51.

Pump 41 according to various embodiments would require a relatively low amount of power, particularly when used for intermittent drug delivery. Short pump duty cycles could also make the presence of implant unit 40 more tolerable to a patient who can sense the vibration of actuator 48 in implant unit 40. Implant unit 40 will (in at least some embodiments) require only a minimal amount of power when operating pump 41. Battery 43 may contain sufficient energy to operate implant unit 40 for up to 30 days on a single charge. While in a standby mode, implant unit 40 battery 43 may lose less then 10% of its full capacity charge in 90 days. As previously indicated, the condition of battery 43 is in some embodiments monitored by electronics 42, which electronics may also monitor the condition of other internal components. Monitored battery conditions can include level of charge, charge and discharge current, temperature, and rate of change of charge during charging and discharging. Battery 43 in at least some embodiments may also be recharged from a state of nearly complete discharge. Electronics 42 may also include a protection circuit to control charging of battery 43 so as to prevent overcharging or charging at an excessive rate, thus also preventing overheating of battery 43 during charging and/or discharging.

Implant unit 40 also includes a housing 45 to support and protect VI pump 41 and other components. Portions of housing 45 that will contact body tissues or drug are formed from biocompatible and/or drug compatible materials. Housing 45 may also incorporate a hermetic enclosure to protect electronics 42 from moisture. In some embodiments, that enclosure is hermetic to 1×10−9 atm-cc/sec or otherwise able to protect internal electronics for the life of the implant. Housing 45 may also incorporate a component for the purpose of absorbing or adsorbing moisture within the hermetic enclosure. Housing 45 also incorporates electromagnetic transparent elements permitting electromagnetic waves to reach coil 44. Housing 45 will remain undamaged through implantation and any normally occurring stressful events (e.g., mild hits and bumps). In certain embodiments the physical size of housing 45 may be less than 10 cm×10 cm×2 cm, and in some embodiments may be 3 cm×3 cm×0.5 cm or smaller. As previously indicated, tubing 49 and 50 facilitates connection of implant unit 40 to catheters, as well as disconnection from such catheters. The catheters may be single or multilumen, and may also incorporate a biocompatible sheath that can envelope implant unit 40 and/or a terminal component.

In certain embodiments implant unit 40 has four operational states: active, standby, communicating and charging (C&C), and shutdown. In the active mode, circuitry controlling communications and charging are shut off. In this mode, electronics 42 generates the voltage supply for actuator 48. When in active mode, pump 41 cycles in accordance with the period and duty cycle programmed into memory 51 as part of data corresponding to one or more dosing sequences. In C&C mode, implant unit 40 is detecting a magnetic field within the resonate frequency band of the coil 44 communication circuit. In this mode, implant unit 40 electronics 42 are fully active, but pump 41 activity is terminated. Electronics 42 monitors voltage of battery 43 and detunes the coil circuitry when appropriate to prevent overcharging. Electronics 42 may also monitor frequency variations in a detected magnetic field and attempt to demodulate and decode a frequency shift keyed (FSK) signal. If an FSK signal is detected, electronics 42 will decode it and verify that it is a command intended for implant unit 40. Only a small command space is required for implant unit 40. Commands may be sent in bursts each lasting 10 mS. Between these bursts may be 90 mS intervals of continuous wave magnetic field. During these intervals, implant unit 40 will load and unload the coil to send telemetry data in response to the commands. Unless a command to resume pump 41 activity is received while the magnetic field is present, implant unit 40 will go to standby mode when a magnetic field is removed.

In standby mode, all systems are shut off. A command from a PIU will put implant unit 40 into standby mode. All power to electronics 42 is shut off, except for power to circuits needed to detect a PIU or charger magnetic field (or needed to periodically activate circuits for detecting a magnetic field). When a magnetic field is detected, implant unit 40 will come out of standby mode and go into C&C mode. If the magnetic field is removed without a command to change from standby mode, implant unit 40 will return to standby after the field is removed. When battery 43 is depleted, implant unit 40 goes to shutoff mode. Shutoff mode is similar to standby mode, except implant unit 40 will not go immediately into C&C mode when a magnetic field is detected. If implant unit 40 went into shutoff mode resulting from a depleted battery condition, implant unit 40 will wait unit a minimal charge is available before going into C&C mode.

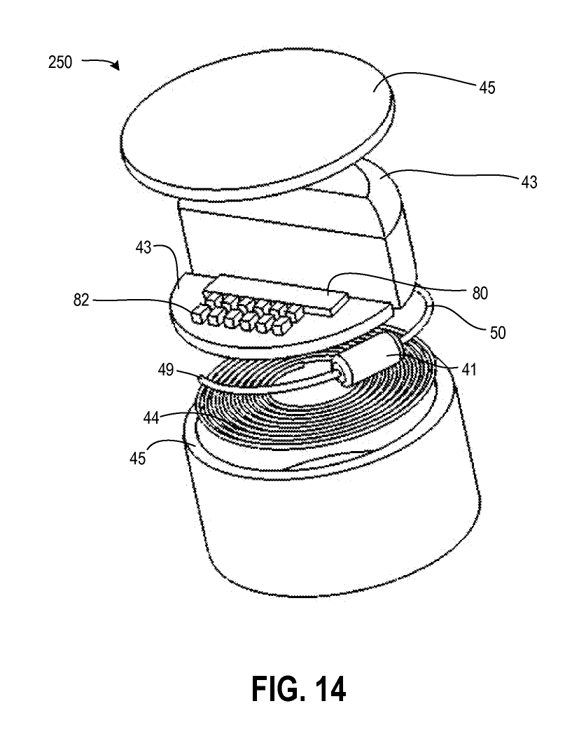

FIG. 14 is an assembly drawing of a physical configuration for an implant unit 40 according to one embodiment. Not shown in FIG. 14 are a catheter (or other conduit) connecting VI pump 41 to a source of bodily fluid (which could be provided to the pump 41 inflow or to a reservoir inflow), a reservoir to hold solid drug (which could be connected to the pump 41 inflow or outflow), a catheter connecting the pump 41 inflow or outflow or a reservoir with a terminal component in a target tissue being treated, or a terminal component. Although a compact cylindrical shape and stacked components are shown in FIG. 14, other embodiments have other shapes, and certain components could be contained in one or more separate housings and connected by wires and/or fluid-carrying elements to a housing containing the pump 41.

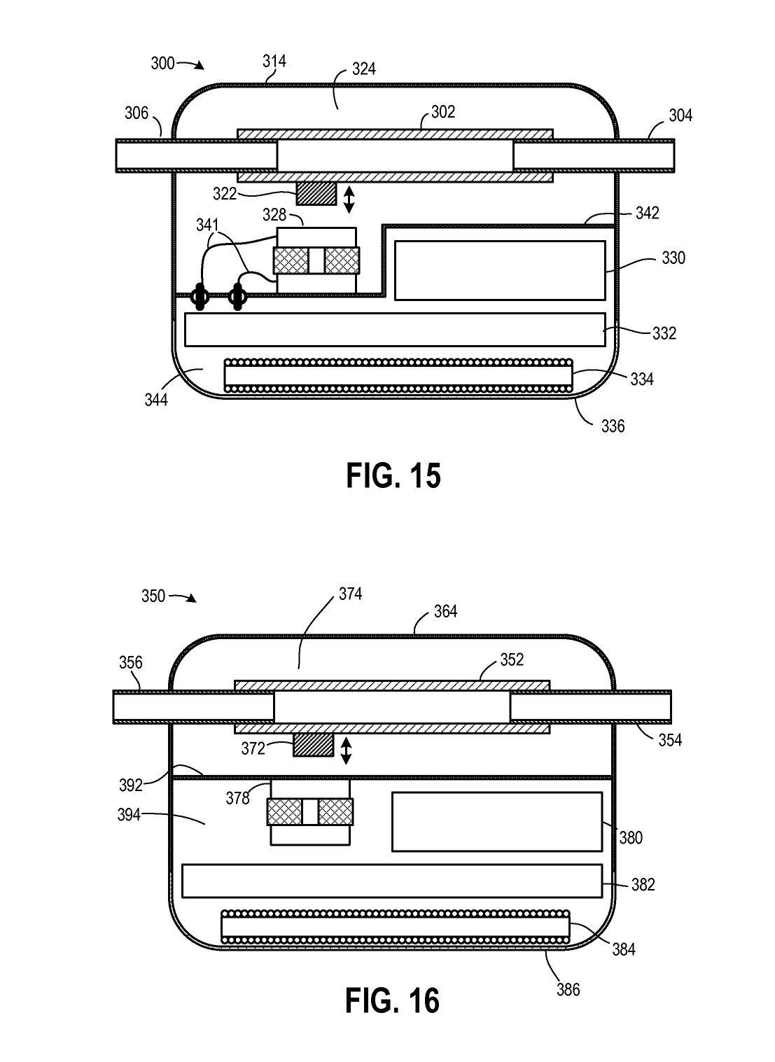

FIG. 15 is a partial cross-sectional view showing an implant unit 300 according to another embodiment. Like implant unit 40 of FIGS. 3 and 14, implant unit 300 can be utilized, e.g., in embodiments according to FIG. 1 or FIG. 2. Implant unit 300 includes an elastic tube 302 (the pump chamber) contained in a rigid, hermetically sealed housing 314. The inlet side of tube 302 is connected to rigid inlet tube 304 and the outlet side of tube 302 is connected to rigid tube 306. Tubes 304 and 306 pass through the walls of housing 314 and are sealed to housing 314 so as to only allow fluid passage through the internal passages of tubes 304 and 306. A permanent magnet or other magnetically-reactive material (e.g., an iron or other ferrous element) 322 is attached to tube 302. An electromagnet 328 is mounted on inner wall 342. Inner wall 342 hermetically seals space 324 (which holds tube 302, magnet 322 and electromagnet 328) from a separate space 344. Space 344, which is also hermetically sealed, contains a circuit board 330 having control and drive electronics for electromagnet 328, battery 332 for powering circuit board 330 and driving electromagnet 328, and a coil and ferrite 334 for charging of battery 332 from a power source that remains external to the patient. Ferrite and coil 334 may also act as an antenna to receive instructions for, e.g., reprogramming circuit board 330; a separate antenna (not shown) could also be included. Electromagnet 328 is connected to circuit board 330 by wires 341 passing through sealed openings in inner wall 342. Space 324 is in at least some embodiments filled with a fluid such as saline or a gelatinous material (e.g., a hydrogel).

Electromagnet 328 is positioned such that magnet 322 (fixed to flexible tubing 302) is in proximity. As current flows through the windings of electromagnet 328, magnet 322 is alternately attracted and repelled by electromagnet 328, and thereby flexing tubing 302 and generating a pumping action. By controlling the rate and magnitude of current through electromagnet 328, the frequency and magnitude of force exerted on tube 302 is controlled, thereby controlling the flow rate through the VI pump formed by tubing 302, tubes 304 and 306 magnet 322.

In at least some embodiments, housing 314 is formed from one or more rigid, biocompatible materials. Examples include metallic materials (e.g., titanium) and ceramic materials (e.g., yttria stabilized zirconia). If metallic materials are used, a separate ceramic “window” 336 can be included so as to permit magnetic flux and RF communications from an external source to reach ferrite and coil 334 (and a separate antenna, if present). Housing 314 can be formed so that the external shape of implant unit 300 fits easily in a desired implantation site in a patient.

FIG. 16 is a partial cross-sectional view showing another example of an implant unit that can be used, e.g., in embodiments according to FIG. 1 or FIG. 2. Implant unit 350 shown in FIG. 16 is similar to implant unit 300 of FIG. 15, except that magnet (or other magnetically-reactive element) 372 of implant unit 350 is moved by an electromagnet 378 on an opposite side of internal wall 392. Internal wall 392, which forms a hermetic barrier between space 374 and space 394, is formed from a material which permits passage of electromagnetic flux from electromagnet 378. This configuration allows electromagnet 378 to be contained within the electronics package and avoids having electromagnet 378 come into contact with fluid. The remaining components in FIG. 16 are similar to components of FIG. 15 having a reference number offset by 50 (e.g., elastic tube 302 of implant unit 300 is similar to and serves the same purpose as elastic tube 352 of implant unit 350, electronics 380 of implant unit 350 are similar to and the serve the same purpose as circuit board 330 of implant unit 300, etc.). Internal space 374 is in at least some embodiments filled with a fluid such as saline or a gelatinous material (e.g., a hydrogel). Housing 364 can be formed so that the external shape of implant unit 350 fits easily in a desired implantation site in a patient.

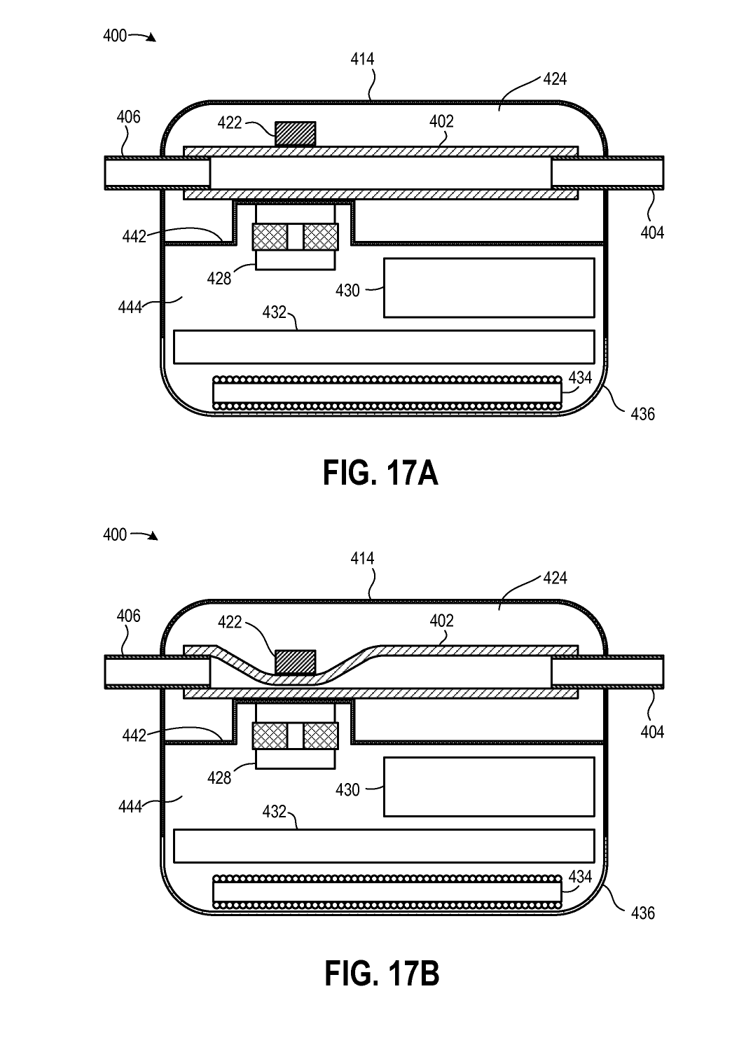

FIGS. 17A and 17B are partial cross-sectional views showing another example of an implant unit that can be used, e.g., in embodiments according to FIG. 1 or FIG. 2. Implant unit 400 shown in FIG. 17A is generally similar to implant unit 350 shown in FIG. 16. Unlike implant unit 350 of FIG. 16, however, the permanent magnet 422 of implant unit 400 is attached to flexible tube 402 so that tube 402 is between permanent magnet (or other magnetically-reactive element) 422 and electromagnet 428. When electromagnet 428 is de-energized, as shown in FIG. 17B, magnet 422 is attracted to the ferrous core of electromagnet 428 and pinches tube 402 closed. A rigid stationary object may be placed next to flexible tube 402 on the opposite side of magnet 422 so as to provide a location against which tube 402 is compressed (in the embodiment shown, inner wall 442 is configured so as to form such a location). In this manner, the flow of vehicle in an implanted drug delivery system can be stopped by turning off the VI pump. Remaining components in FIGS. 17A and 17B are similar to, and perform similar functions as, components in FIG. 16 having reference numbers offset by 50 (e.g., battery 432 of FIGS. 17A and 17B is similar to and performs a similar function as battery 382 of FIG. 16).

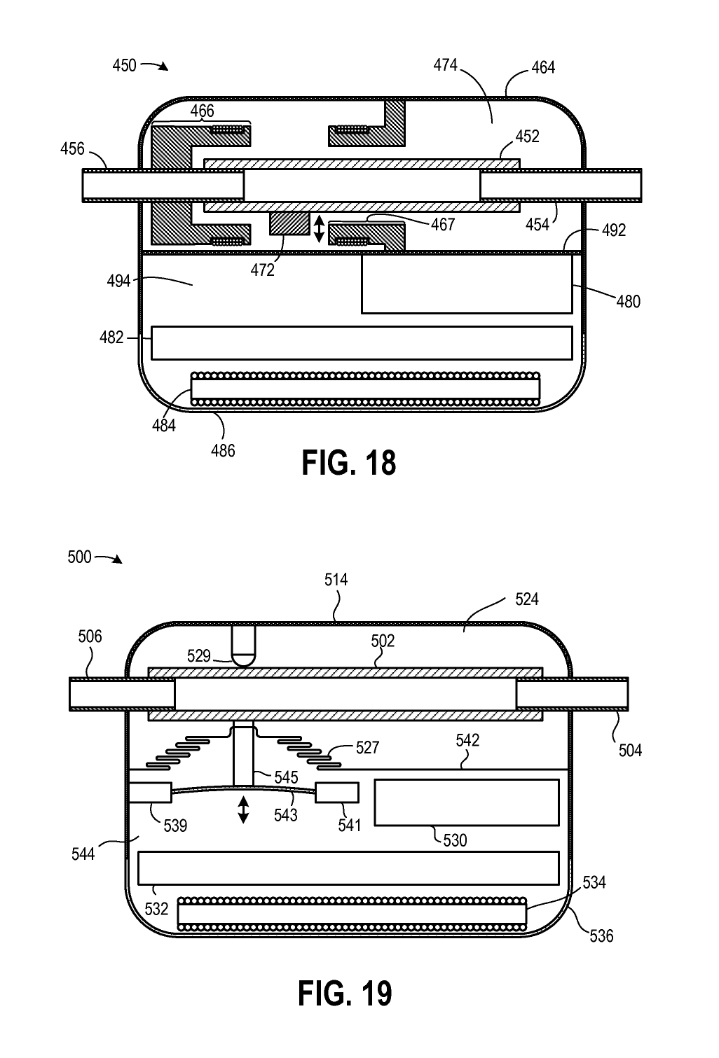

FIG. 18 is a partial cross-sectional view showing another example of an implant unit that can be used, e.g., in embodiments according to FIG. 1 or FIG. 2. Implant unit 450 shown in FIG. 18 is also similar to implant unit 300 of FIG. 15, except that internal space 474 of implant unit 450 includes a first ferrite tube and coil structure 466 surrounding elastic tube 452 near the outlet end and a second ferrite tube and coil structure 467 surrounding tube 452 and that is closer to the inlet end. Permanent magnet (or other magnetically-reactive element) 472 is attached to tube 452 midway between structures 466 and 467. Structures 466 and 467 are connected to electronics 480 and to each other by wires (not shown).

Magnet 472 is oriented such that when the coils of structures 466 and 467 are energized, the magnetic field gradient causes magnet 472 to move inward toward the center of tube 452, thus compressing tube 452. The ferrite tubes of structures 466 and 467 hold (and are surrounded by) the coils through which current flows. The ferrite tubes will support the coils in locations proximate to magnet 472 while at the same time allowing tube 452 to move. The ferrite tubes also help direct the magnetic flux created by the coils such that magnet 472 is displaced using less energy than would be required if the coils were wound directly onto tube 452.

The remaining components in FIG. 18 are similar to and perform similar functions as components of FIG. 15 having a reference number offset by 150. Internal space 474 is in at least some embodiments filled with a fluid such as saline or a gelatinous material (e.g., a hydrogel). Housing 464 can be formed so that the external shape of implant unit 450 fits easily in a desired implantation site in a patient.

FIG. 19 is a partial cross-sectional view showing another example of an implant unit that can be used, e.g., in embodiments according to FIG. 1 or FIG. 2. Similar to the embodiments of FIGS. 15-18, implant unit 500 of FIG. 19 includes a housing 514 having a magnetically transparent portion 536, a flexible tube fluid chamber 502, rigid inlet and outlet tubes 504 and 506, battery 532, electronics 530, and a ferrite and coil 534. Unlike the embodiments of FIGS. 16-18, however, the VI pump actuator of implant unit 500 employs a flexing piezoelectric element 543 attached to two supports 539 and 541. Supports 539 and 541 are attached to housing 514. A post 545 attached to element 543 moves upward against tube 502 when a voltage is applied to element 543. A corresponding fixed pincher element 529 can be located on an opposite side of tube 502. Hermetic barrier 542 separates space 524 containing tube 502 from space 544 containing electronics and other elements, and includes a flexible bellows portion 527.

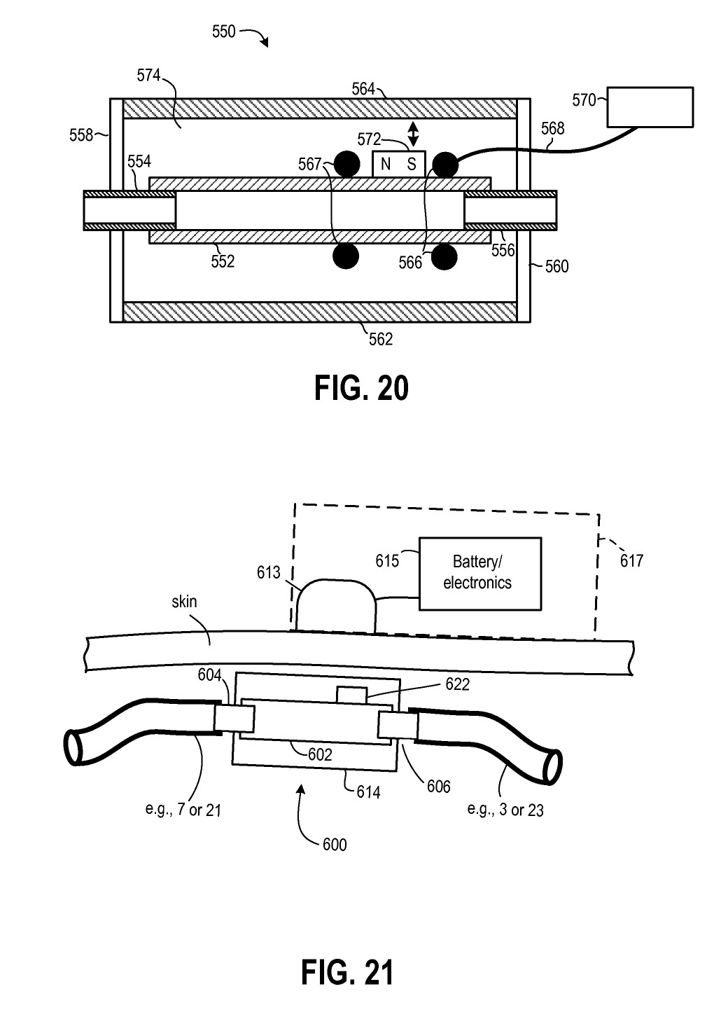

FIG. 20 shows another example of an implant unit that can be used, e.g., in embodiments according to FIG. 1 or FIG. 2. Unlike the embodiments of FIGS. 15-19, however, implant unit 550 of FIG. 20 does not include control electronics, a battery, or a communication/charging coil. Instead, those elements are contained in a separate implant unit 570 that is connected to implant unit 550 by wires 568. Implant unit 550 includes an elastic tube 552 contained in a rigid, hermetically sealed housing 564 to protect tube 552 from external forces. Actuating tube 552 is coupled at an inlet end to a first connector tube 554 and at an outlet end to a second connector tube 556. Connector tubes 554 and 556 are in some embodiments rigid (i.e., substantially less elastic than tube 552). Connector tubes 554 and 556 extend through (and are sealed to) end caps 558 and 560, respectively. End caps 558 and 556 are in turn attached to body member 562. Caps 558 and 560 and body 562 form a sealed housing 564 in which fluid may only enter or leave through internal passages of connector tubes 554 and 556.

Inductive coils 566 and 567 are wound around tube 552 and connected by wires 568 to actuating electronics and a power source (e.g., one or more lithium-ion batteries) contained in separate implant unit 570. Wires 568 pass through an opening in cap 560, with the opening sealed to prevent incursion of bodily fluids inside housing 564. A permanent magnet (or other magnetically-reactive element) 572 is glued to tubing 552 between coils 566 and 567. Magnet 572 is positioned generally equidistant from coils 566 and 567 and oriented so that the axis of its north and south poles are aligned parallel to tube 552. Current simultaneously pulsed through coils 566 and 567 forms a magnetic field generally centered on the central longitudinal axis of tube 552. Permanent magnet 572 attempts to align itself with the generated magnetic field and moves radially inward toward the center of tube 552. By controlling the rate and magnitude of current pulsations through coils 566 and 567, the frequency and magnitude of force exerted on tube 552 is controlled, thereby controlling the flow rate through pump implant unit 550. Although the embodiment of FIG. 20 shows separate coils 566 and 567, a single coil extending over the ends of permanent magnet 572 can be used. Alternatively, multiple coils on both ends of permanent magnet 572 can be used. As yet another alternative, one or more coils such as coils 566 and 567 and a permanent magnet attached to tube 552 can be configured so that energizing the coil(s) causes the permanent magnet to move radially outward from the tube.

Components of housing 564 (body member 562 and end caps 558 and 560) may in at least some embodiments be formed from one or more rigid, biocompatible materials. Examples include metallic materials (e.g., titanium) and ceramic materials (e.g., yttria stabilized zirconia). If metallic materials such as titanium are used, end caps 558 and 560 may be laser welded to element 562. The internal space 574 between housing 564 and tube 552 is in at least some embodiments filled with a fluid such as saline or a gelatinous material (e.g., a hydrogel). Rigid connecting tubes 554 and 556, which may be made of a biocompatible material such as titanium, create a reflection site which causes fluidic wave reflection. Tubes 554 and 556 may, depending on material choices for those tubes and for end caps 558 and 560, be laser-welded to the housing to provide a hermetic seal. Sealing of housing 564 prevents incursion of bodily fluids into space 574 and interfering with the operation of implant unit 550. For example, internal components of implant unit 550 may be formed from materials which are not biocompatible, and incursion of body fluids could result in formation of deposits that would hinder pump operation or diffuse into tube 552 and affect drug concentration. Although housing 564 is cylindrical in shape, other shapes may be used so as to form a pump housing that fits easily in an implantation site on the side of a patient's skull or in another body location.

In still other embodiments, electronics and an inductive coil for moving a permanent magnet or other magnetically-reactive material (attached to a VI pump chamber) remain external to the patient. FIG. 21 is a block diagram of some such embodiments. In the embodiment of FIG. 21, an implant unit 600 contains a VI pump chamber 602 (e.g., a flexible tube or chamber with a flexible membrane) attached to rigid inlet and outlet 604 and 606. A permanent magnet 622 is attached to a flexible wall of chamber 602. An inductive coil 613 is external to the patient and is used to move permanent magnet 622. Control electronics and a power source (e.g., a battery) can be contained in a separate unit 615, or coil 613 and electronics/power source 615 could be contained in a single housing 617 (e.g., within a PIU). Housing 614 of implant unit 600 is formed from a biocompatible, nonconductive material (e.g., yttria stabilized zirconia) that permits magnetic flux to pass, but which provides sufficient rigidity to support and protect the internal components of implant unit 600. Implant unit 600 could be employed, e.g., in embodiments according to FIG. 1 or FIG. 2, with inlet 604 coupled to a catheter in fluid communication with a vehicle source (e.g., catheter 7 of FIG. 1 or catheter 21 of FIG. 2) and outlet 606 coupled to a catheter in fluid communication with a drug reservoir (e.g., catheter 3 of FIG. 1 or catheter 23 of FIG. 2).

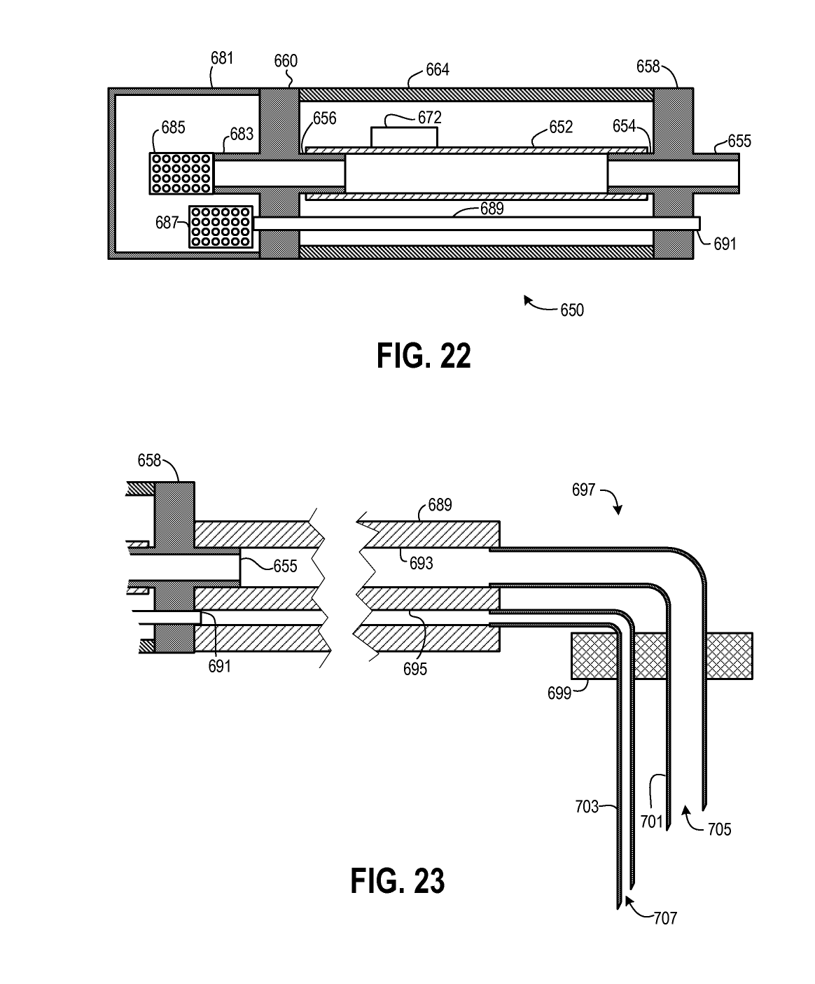

FIG. 22 is a cross-sectional view of an implant unit 650 according to another embodiment. As with implant unit 600 of FIG. 21, implant unit 650 relies on a magnetic field from an external source (e.g., a PIU) to move a magnetically-reactive force-transferring member attached to a VI pump chamber. Implant unit 650 includes a cylindrical outer housing 664 formed from a material that will permit passage of magnetic flux (e.g., yttria stabilized zirconia or sufficiently thin walled titanium). A rigid first end cap 658 is sealed to housing 664 and includes an inlet 655 and an outlet 691 of a tube 689. An internal side of end cap 658 includes an inlet rigid attachment point 654 for flexible tube 652. A second rigid end cap 660 includes an outlet rigid attachment point 656 for tube 652 and an outlet 683 on the opposite side. Tube 689 similarly passes through end cap 660; tube 689 is sealed to end caps 658 and 660 to prevent leakage into or out of the inner volume of housing 664. A permanent magnet (or other magnetically-reactive element) 672 is attached to flexible tube 652. A second housing 681 is sealed to the outer face of end cap 660 to form a drug reservoir. A first screen 685 may be attached to the opening of outlet 683 and a second screen 687 may be attached to an opening at the end of tube 689. In some embodiments, cylinder 664 is formed from a ceramic and includes biocompatible metal rings (not shown) brazed to its ends, thereby permitting welding of end caps 658 and 660 to housing 664.

In operation, a vehicle is drawn through inlet 655 and flows into the drug reservoir formed by housing 681. Drug-laden vehicle then passes out of implant unit 650 through outlet 691. Implant unit 650 could be employed, e.g., in embodiments according to FIG. 1 or FIG. 2, with inlet 655 coupled to a catheter in fluid communication with a vehicle source (e.g., catheter 7 of FIG. 1 or catheter 21 of FIG. 2) and outlet 691 coupled to a catheter in fluid communication with a terminal component (e.g., catheter 5 of FIG. 1 or catheter 25 of FIG. 2). FIG. 23 is a cross-sectional view showing use of implant unit 650 with a dual lumen catheter 689 in fluid communication with a terminal component 697 that also serves as a vehicle inlet. Specifically, a terminal component in the form of a double needle 697 includes a first needle 703 positioned to withdraw a bodily fluid through inlet 707 and a second needle 701 positioned to discharge drug-laden bodily fluid through an outlet 705, with inlet 707 and outlet 705 offset from one another. Double needle 697 may also include an insertion stop 699. The internal passage of first needle 703 is in fluid communication with a first lumen 695 of catheter 689. The internal passage of second needle 701 is in fluid communication with a second lumen 693 of catheter 689. Although FIG. 23 shows needle 703 having smaller inner and outer diameters than needle 701, the reverse could be true, or needles 701 and 703 could be of the same size.

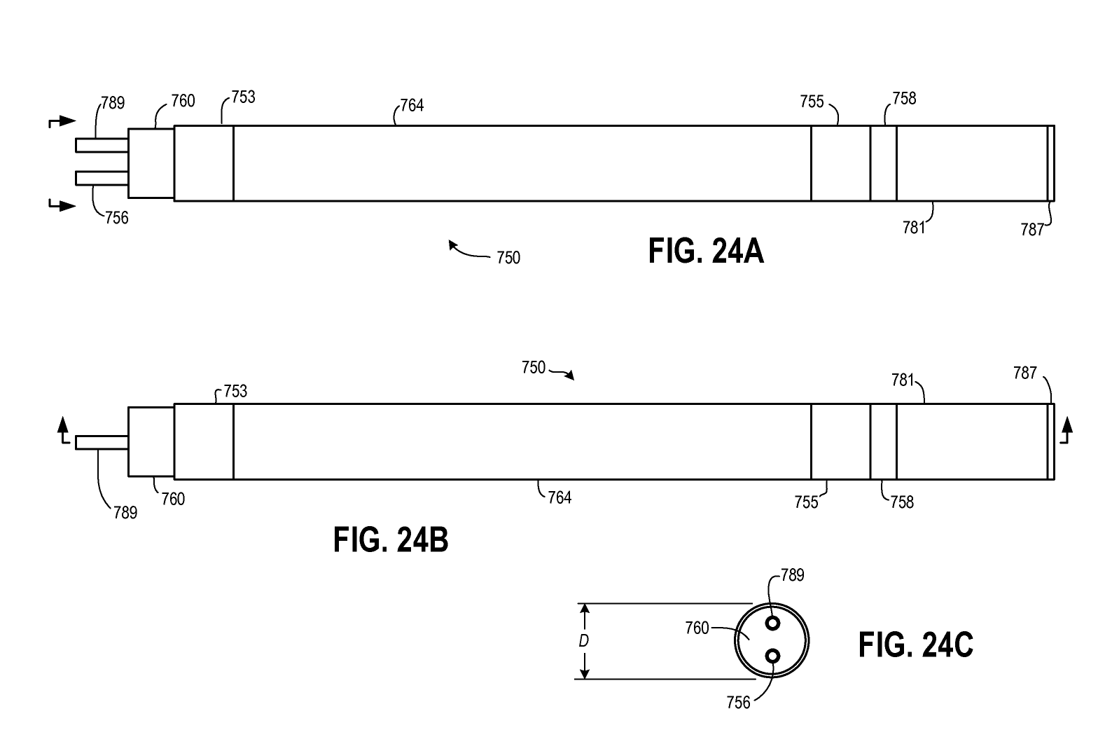

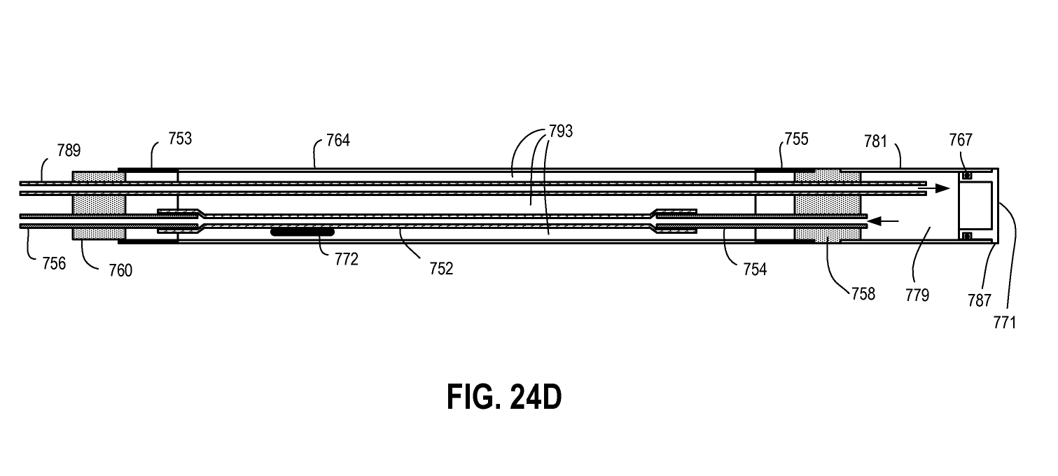

FIGS. 24A-24D show a variation on the embodiment of FIG. 22. FIGS. 24A and 24B are top and side views, respectively of implant unit 750. FIG. 24B is a front view from the location indicated in FIG. 24A. FIG. 24D is a cross-sectional view of implant unit 750 from the location shown in FIG. 24B. Implant unit 750 is similar to implant unit 650 of FIG. 22, but has a longer and thinner profile. In some embodiments, implant unit 750 has a maximum outer diameter D of approximately 3 to 10 mm and a length of approximately 30 mm. Implant unit 750 includes a cylindrical outer housing 764 formed from a material that will permit passage of magnetic flux (e.g., yttria stabilized zirconia, alumina, titanium). If housing 764 is formed from yttria stabilized zirconia or another other ceramic, ferules 753 and 755 (formed from titanium or other biocompatible metal) are brazed onto the ends to facilitate laser welding of end caps 760 and 758. If housing 764 is formed from titanium, end caps 760 and 758 may be laser welded directly to housing 764.

A rigid first tube 789 passes through end cap 760, through the interior 793 of housing 764, and through end cap 758 into a drug reservoir volume 779 formed by a titanium drug reservoir housing 781. Housing 781 is laser welded to end cap 758. The outer edges of tube 789 are sealed (e.g., by laser welding) to end caps 760 and 758 to prevent leakage into or out of housing interior 793 or reservoir volume 779. The outer edges of rigid tubes 756 and 754 are similarly sealed to end caps 760 and 758. A VI pump chamber in the form of flexible tube 752 is attached at one end to rigid tube 756 and at the other end to rigid tube 754. A permanent magnet 772 is attached (e.g., with silicone or other adhesive) to flexible tube 752. Magnet 772 (or alternatively, another magnetically-reactive material) may also be encapsulated in silicone or other material so as to prevent contact between magnet 772 and liquid filler material (e.g., hydrogel) filling interior space 793 of housing 764.

End cap 787 attaches to reservoir housing 781 and forms a rear wall of a drug reservoir. In some embodiments, end face 771 of end cap 787 may include an elastomeric septum to facilitate injection of fluid into volume 779. In some embodiments, end face 771 may incorporate a membrane (e.g., a hydrophobic biocompatible material such as PTFE) that allows migration of air bubbles from reservoir volume 779. An O-ring 767 seals reservoir volume 779. In some embodiments, end cap 787 may include clips (not shown) to hold cap 787 in place.

As seen in FIG. 24D, end cap 758 includes a ridge acting as a stop for ferrule 755 and as a stop for reservoir housing 781. This permits correct location of internal VI pump components (tubes 756, 752 and 754 and magnet 772) during assembly. End cap 760 has a profile that fits within ferrule 753 (or within housing 764 if ferrule 753 is not used). This profile of end cap 760 permits assembly and testing of the VI pump components prior to assembly of those components into housing 764.

Implant unit 750 can be used, e.g., in embodiments according to FIG. 1 or FIG. 2, with tube 789 coupled to a catheter in fluid communication with a vehicle source (e.g., catheter 7 of FIG. 1 or catheter 21 of FIG. 2) and tube 756 coupled to a catheter in fluid communication with a terminal component (e.g., catheter 5 of FIG. 1 or catheter 25 of FIG. 2). Implant unit 750 can also be used with multi-lumen catheters (e.g., in a manner similar to that described above in connection with claim 23). In some embodiments, an implant unit similar to implant unit 750 may be configured such that the VI pump receives vehicle through one opening in the implant housing and pushes the vehicle into the drug reservoir volume and out of another housing opening. As with implant units 600 and 650, implant unit 750 relies upon magnetic flux from an external source (e.g., a PIU) to cause movement of magnet 772. The low profile of implant unit 750 permits implantation using laparoscopic and other minimally-invasive techniques. A ridge or other feature can also be added to the external surface of implant unit 750 to facilitate proper location within a patient's body. In some embodiments, tube 789 can be replaced with a second VI pump similar to the VI pump formed by tubes 756, 752 and 754 and magnet 772, thereby providing an implant unit with two pumps in series to increase output pressure.

Further embodiments include additional variations on the implant units described above. Rather than a flexible tube (e.g., tube 302, 352, 402, 452, 502, 552, 602, 652 or 752), a valveless impedance pump may employ a thin flexible membrane (coupled to a rigid surrounding material) in direct contact with the fluid pathway and an actuator which vibrates the membrane at an asymmetric location along the length of the membrane. An actuating magnet can be encapsulated with a biocompatible material such as a ceramic or polymer (e.g., a fluoropolymer) to prevent contact between the magnet and surrounding fluid. A rigid stationary object may be placed on the other side of a flexible tube to oppose a magnet (or other pinching element) and provide a location against which the tube is compressed. Instead of a magnetically-reactive force-transferring member compressing an elastic actuating tube, a piezoelectric element could be employed. Some embodiments may employ a plurality of pinching elements located along the length of a flexible tube. Using multiple pinching elements, a peristaltic effect can be initiated to create flow in one direction by activating the pinching elements in cascading succession along the length of the flexible tube. Other configurations can be used to create inlet and/or outlet connections suitable for multi-lumen tubing. An implanted pump can be operated so as to deliver drug to a target tissue on an intermittent or continuous basis. A pump can also be configured so that the permanent magnet or other force-transferring member is compressing the flexible actuating tube when power is not applied to coils or other energizing elements, with the permanent magnet or other force-transferring member moved away from the flexible tube centerline (thus decompressing the tube) when the coils or other energizing elements are powered.

In some embodiments, a flexible circuit board can be used to hold and connect the elements of an implant unit electronics (e.g., electronics 42 of FIG. 3). Flexible circuit boards can similarly be used in a PIU or other external component of a drug delivery system. A communication and charging coil can also be fabricated into such a flexible circuit board by routing coil traces around the periphery of the board in order to increase coil diameter. Those traces can then be partially cut and folded away from the rest of flexible circuit board. Other traces in the flexible circuit board can be routed either distant from the coil traces or perpendicular to the path of the coil conductor so as to reduce inductance from the coil into other circuits. Additional small inductors can also be created within the flexible circuit board for use within separate circuits not intended to interact with electromagnetic fields of other circuits. These small inductors can also be partially cut from the flexible circuit board and folded away from the plane of the larger coil so as to minimize the induction from the large coil into the small inductor.

Components mounted to a flexible circuit board can include any chips, discrete components or connectors. The flexible circuit board can be located within the device such that the circuit is located adjacent to an electromagnetically transparent barrier, thereby allowing a charging/communication coil to interact more efficiently with an external device. In some embodiments, a flexible circuit board may include a coil used to create the magnetic flux used to induce motion in a pump force-transferring member (e.g., magnet 722 in FIG. 24D).

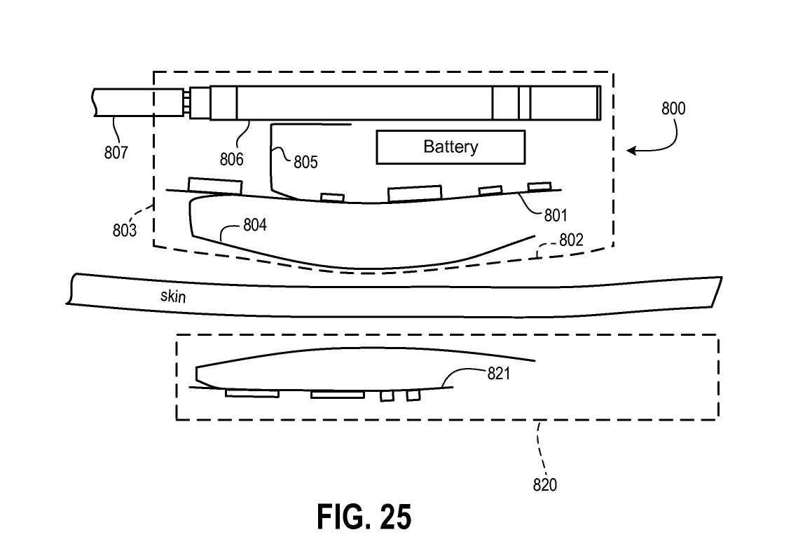

FIG. 25 shows one example of an implant unit 800 that includes a flexible circuit board 801 located adjacent to a magnetically-transparent window 802 in a housing 803. Flexible circuit board 801 includes a large communication/charging coil 804 and a second coil 805 providing the magnetic flux to move a force-transferring member within a pump/reservoir unit 806. Pump/reservoir unit 806 may be an implant unit (such as implant 650 of FIG. 22 or implant unit 750 of FIGS. 24A-24D) that is itself contained within housing 803, with a dual lumen catheter 807 passing through housing 803 to reach pump/reservoir unit 806. As also shown in FIG. 25, a PIU 820 can include electronics and a communication/charging coil mounted onto a flexible circuit board 821.

### System Components External to the Patient

In addition to components that are implanted in a patient's body, systems according to some embodiments include components that remain external to the patient' body. In at least some embodiments, a patient interface unit (PIU) is used to communicate with an implant unit located inside a patient's body. The PIU can also communicate with a computer on which physician interface software is executed. A separate charging unit can also be used to charge an implanted implant unit.

After a pump-containing implant unit has been placed into a patient body, a PIU is used to activate, deactivate and otherwise control the implant unit. The PIU can communicate with the implant unit, upload instructions to the implant unit, download data from the implant unit (e.g., dosing data related to pump actuation times, status data for components of the implant unit), and (in some embodiments) charge or partially charge the implant unit. Commands that might be sent from a PIU to an implant unit include, but are not limited to, commands instructing the implant unit to resume drug delivery operations, to increase drug delivery duty cycle, to decrease drug delivery duty cycle, to respond with current drug delivery duty cycle, to respond with implant unit battery power level, to stop drug delivery operation, to continue operation—send communication acknowledge, to respond with an implant unit ID, etc. A PIU could also be programmed to enforce limitations on maximum or minimum parameters that are allowed for the implant unit (e.g., maximum drug delivery duty cycles or maximum duration for a sequence of events), and attempts to exceed such limits with the transmission of a conflicting command could result in an audible alarm sounding or flashing of a display (and/or refusal to enter the conflicting command into a command queue such as is described below). In some embodiments, violations of preset limits may be allowed by inputting a password or inserting a physical key into the PIU. In some embodiments where an implant unit relies on an externally applied magnetic field to move a VI pump force-transferring member (e.g., as in FIGS. 21-24D), a PIU can also be used to supply the necessary magnetic flux.

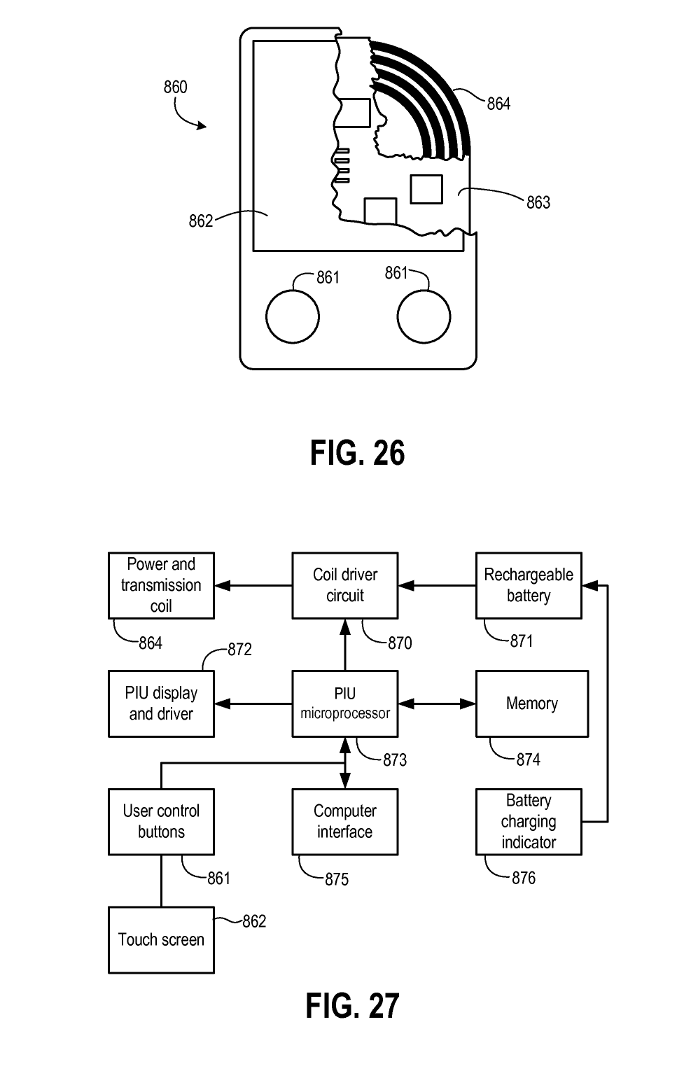

FIG. 26 is a front view of a handheld PIU 860 according to some embodiments. PIU 860 is powered by a rechargeable and/or replaceable battery. A display screen 862 provides information to a user concerning status of an implant unit or of PIU 860. One or more keys 861 are used to cycle through PIU menus and otherwise provide user input. Keys 861 may be soft keys having multiple functions that depend on the operational state of PIU 860. A portion of the housing of PIU 860 and of display screen 862 is removed in FIG. 26 to expose an internal circuit board 863 containing electronics of PIU 860. As previously indicated, circuit board 863 could be a flexible circuit board. A portion of circuit board 863 is also removed so as to show a portion of coil 864. Coil 864 is used to create a magnetic field used to communicate with and/or charge an implant unit, to provide magnetic driving force for implant units that rely upon an external driving magnetic field, and to receive communications from an implant unit.

FIG. 27 is a block diagram of internal components of PIU 860. As indicated above, coil 864 produces an AC magnetic field that will inductively couple to a coil in an implant unit. This signal may be FM modulated to transmit commands and data to an implant unit. Coil driver circuit 870 provides the voltage and current necessary to cause the coil 864 to produce the necessary AC magnetic field. In applications where data transmission is required, this circuit will also convert the data stream into the appropriate modulation of the AC field. PIU microprocessor 873 controls all operations of PIU 860. Memory 874 includes volatile (e.g., RAM) and nonvolatile (e.g., FLASH) components, and may include read-only memory. Nonvolatile memory stores operational constants, calibration values and device identification values (e.g., passwords recognized by an implant unit). Nonvolatile memory may also store text data to be displayed on display screen 862, which display screen may be a touch-sensitive screen. The volatile memory is used for calculations and stores intermediate results. When connected to an external computer via interface 875, and after the appropriate password has been received, constants (and/or other data) stored in nonvolatile memory of PIU 860 may be changed. New values can be calculated by the PC support software. PIU 860 may further include other components (not shown) such as a coil impedance sensing circuit, a low level communications control circuit, a button sensing and bounce control circuit, an audible alarm and/or vibrator, a power connector, and power regulation and distribution circuitry.

PIU 860 and an implant unit can be programmed so that a patient can alter the implant unit pump frequency and/or duty cycle corresponding to one or more dosing sequences so as to adjust drug delivery volume and time. PIU 860 can also be connected (e.g., by a USB cable and interface 875) to a computer executing physician interface software, thereby allowing the physician to program the PIU and/or download data from the PIU. The downloaded data may include, e.g., a record of patient use of the PIU and implant unit over a given period of time. With such a record, the physician (using the physician interface software) could then monitor and/or adjust treatment.

Display 862 of PIU 860 may also show charge level of PIU battery 871, or while charging it may show the time until full charge is reached. Display 862 may also flash to alert a patient or other user that an action is required. Display 862 could optionally be a touch screen allowing software navigation with a finger or stylus.

A physician can program PIU 860 to enforce limits on dose frequency and/or dose volume. For example, PIU 860 may be programmed to only allow the implant unit to operate with specified minimum periods between dosing. In these situations, display 862 may show time until the next permitted dose.

PIU 860 may also contain a real-time clock (RTC) which, in some embodiments, can only be set or changed by instructions received via computer interface 875. PIU 860 may in some embodiments record implant unit start and stop times, duty cycle, and changes in duty cycle initiated by the patient. PIU 860 may store this data and permit access thereto via computer interface 875. In addition to monitoring the drug delivery operation, PIU 860 could use this information to calculate implant unit battery level or other implant unit parameters (e.g., drug content remaining) The time in operation and the duty cycle of an implant unit pump can allow PIU 860 to alert the patient when the implant unit battery should be recharged. A short burst audible alarm or short vibration period from PIU 860 could be used to alert the patient of a condition requiring attention.

When PIU 860 is held against a patient's skin, in line with an implant unit, the magnetic field from coil 864 will communicate with the implant unit. In some cases, PIU 860 may be programmed such that it must be used to initiate each dosage pumped by the implant unit. In other cases, an implant unit may be programmed to automatically dispense drug dosages at predetermined intervals or in response to implanted sensors, with PIU 860 mainly used to monitor the implant unit and/or shut down the implant unit. In some embodiments, the signal between coil 864 of PIU 860 and the coil of an implant unit can be used to determine if the alignment of PIU 860 and the implant unit is correct. If a signal detected by PIU 860 is strong enough, a tone or vibration can be emitted to notify the patient of proper alignment.

Nonvolatile memory in PIU 860 may in some embodiments record instructions sent to an implant unit and/or time spent charging, and log communication errors. With stored data regarding hours of implant unit operation, PIU 860 can calculate the appropriate time to recharge the implant unit battery and alert the patient. Using PIU 860, the patient can change the frequency and duration of drug delivery or other dosing sequence parameter(s). PIU 860 will in some embodiments only allow variations of these parameters that are within limits set by a physician. Information stored by PIU 860 can also be available to the physician to provide a more complete therapeutic treatment history. With special commands (that can in some embodiments only originate in the physician interface software), the values in nonvolatile memory of PIU 860 can be reprogrammed.

The patient will operate PIU 860 by selecting a command from a menu. These commands may, e.g., activate the implant unit, cause the duty cycle or period of drug delivery to increase or decrease, or cause the implant unit to go into a hibernate state (e.g., standby mode). PIU 860 is designed for handheld operation and can be relatively small in size. A patient can hold PIU 860 so that display 862 can be easily seen and buttons 861 (and/or additional buttons) operated. Various user interface schemes can be used. For example, a PIU could have one button per command, or the commands could be selected from a pull down menu. Other schemes involving cursors or touch screens could also be used. When a series of commands is to be sent to an implant unit, a patient could in some embodiments enter those commands sequentially and place them in a queue. In some embodiments, a PIU may have 5 buttons to control all operations. Four arrow keys can control menus on the display. Horizontal arrow keys can select a type of command to be sent and vertical arrow keys can scroll up and down through menus to select commands. Once a command is selected it can be added to a queue of commands to be transmitted to an implant unit. Certain commands may also allow queue editing. Such commands may not be part of the transmission space, but may be useful in setting up a list of commands for transmission. Horizontal keys may also be used to select from top level menus and vertical keys may be use to delete, reorder or insert commands in a queue. A select button can be used, e.g., to initiate a transmission and reception sequence. In addition to loading commands into a queue for transmission to an implant unit, arrow keys and pull down menus could also be used to control other aspects of PIU operation. For example, a PIU could also have commands that include, but are not limited to, commands toggling an audible alarm and/or vibrator, a command turning on backlighting of an LCD display, a command to display PIU battery status, and a command to show time before an implant unit requires recharge. The display can be limited in size, but use large letters to allow easy reading by patients.

Once a command is selected from a menu of PIU 860, the patient will place PIU 860 against the skin near the implant site and press a button or otherwise provide user input corresponding to an instruction to commence communication with an implant unit. Alternately, an automatic sensor could determine that proximity to the implant unit is achieved and the commands automatically sent. When the transmit button is pressed, PIU 860 will generate a magnetic field using coil 864. After sufficient time to allow the implant unit to detect continuous wave or carrier wave magnetic field from PIU coil 864, PIU 860 will begin burst FSK modulation consistent with the instruction(s) to be sent. Between burst transmissions, PIU 860 can monitor the load on the magnetic field of coil 864 in anticipation of a response from the implant unit. If the return signal is an “acknowledge,” PIU 860 need not retransmit the signal. In some embodiments, and as described below, PIU 860 provides the carrier wave for both uplink and downlink transmission, and no synchronization if either PIU 860 or of the implant unit is required. In this way, bidirectional communications are achieved with only a single transmitter.

When communications are initiated, coil driver circuit 870 is activated and energy from battery 871 charges a resonant LC circuit in coil driver circuit 870. As a result the magnetic field of coil 864 builds, collapses, rebuilds with the opposite polarity and again collapses. This process repeats at a rate of, e.g., 127 KHz, or higher rate depending on the specific implementation, so that the frequency is much higher than data rates and within a frequency band not restricted by local communications agencies. The implant unit will sense this signal and recognize it as center frequency. Shifts to slightly higher frequencies can be designated as logical ones and shifts to lower frequency can be received as logical zeros. Mark and space schemes may be used to simplify the demodulation process. Other modulation schemes may be used. To reduce power consumption of the implant unit, communications can be restricted to narrow bandwidths. This is easily accomplished if the channel capacity is limited, which is in turn easily accomplished in situations where a maximum baud rate is kept low.

The number of bits in PIU communication is not limited, but one implementation could use as few as 8 bits. Complex inscription could be added to the PIU and to the implant unit, or may be eliminated for simplicity. In simpler implementations, each command could have a Hamming distance of 3, and hence require at least 3 errors to result in a misread command. In other implementations, some commands may be given a higher Hamming distance and less important commands be given lower Hamming distance. This approach would give very low probability of critical errors and higher probability of errors with inconsequential results. If the received data pattern does not correspond to one of the patterns associated with a command, the pattern can be rejected as an error.

After a data byte is received from PIU 860, an implant unit can wait a fixed interval and then begin sending the response. In one implementation the response may take the form of asynchronous amplitude shift keyed data generated by changing the impedance on the implant coil. One method of performing this would be to short or detune the implant unit coil at the start of a cycle, when the current in the implant unit coil (e.g., coil 44 of FIG. 3) is zero. Because such a detune/short capability may be present in the implant unit charging coil subsystem to prevent over charging, utilizing such capability for simple communications adds functionality without adding potential points of failure. An implant unit may also disconnect a resonant capacitor and connect a low resistance (e.g., zero Ohms) across its coil.

Alternately, an implant unit battery could be used in cases when charging is required. PIU 860 would note an increase in the current load on the magnetic field and register a data bit, the first of which is recorded as a start bit. This change in load could be registered as a logical zero and used to synchronize a receiver clock. At one symbol time later the implant unit may either short or open the coil circuit, and the PIU could then register either a logical 1 or a logical 0, respectively.

In some embodiments a response from an implant unit could be as few as 10 bits (e.g., a start bit, a CRC end bit and 8 data bits). The 8 data bits may contain telemetry information, may be an acknowledgment of a received command, or an indication that a received command was logged as an error. If longer strings of data are required, multiple frames could be used, or varying length transmissions could be designed into the system with only modest increases in complexity. Telemetry can be transmitted several times and compared to verify that a correct value was received. This approach can drive the probability of error closer to zero.

As indicated above, PIU 860 can be used to activate an implant unit and to set the frequency, duty cycle and other parameters of a dosing sequence. This information can be stored in the nonvolatile memory of PIU 860. With this information, PIU 860 can estimate when recharging is appropriate to optimize battery life. An audible alarm that lasts, for example, 3 seconds and a flashing display backlight that lasts, for example, 10 seconds can alert the user that charging is appropriate. To reset the implant charge timer, the patient can complete a charging period and use PIU 860 to communicate with the implant unit to verify full battery charge.

PIU 860 will in some embodiments produce a magnetic field that will be sufficient to transfer charging energy to an implant unit battery, although at a slow rate. In some embodiments, a system includes a separate charging unit that is used for charging the implant battery at a faster rate. The implant unit charging unit can be a transportable unit that uses wall plug power. During the charging process, the charging coil of the charging unit is held in place adjacent to the implant unit (e.g., placed on the skin of the patient's body over the implant unit location). Full charge of the implant unit battery should require approximately 20 minutes. In some embodiments, it is recommended that the implant unit battery not be allowed to drop below 75% of full charge. If charge is maintained at this level, charging should require approximately 5 minutes. In some embodiments, the charging process is open loop, and the implant unit battery level is not monitored during the charging process and communications do not take place. While charging an implant unit, PIU 860 may be connected to the charging unit to monitor charging time and update the expected implant status.

In some embodiments, PIU 860 includes external power connector permitting connection of PIU 860 to an external transformer to draw low voltage power from a wall socket. Such an interface would require only a single unregulated DC voltage supply. Different voltage levels as required by the internal circuitry of PIU 860 could be created, regulated and filtered as needed by the power regulation and distribution circuitry. This approach could prevent a patient from putting high voltage in contact with his or her skin while PIU 860 is operational. When the external power source is connected, microprocessor 873 would recognize the condition and switch from battery 871 operation to charging. In some embodiments, PIU 860 would not be able to communicate with an implant unit during the charging operation, and display 862 would show the current battery 871 energy level, with a buzz or beep indicating that charging of battery 871 is complete. Alternately, PIU 860 could be completely deactivated during all charging operations.

Low power design of PIU 860 can reduce the frequency of required recharging. For example, some sections of PIU 860 can be shut down when not in use. Coil 864, driver circuit 870, a resonant coil driver, an impedance sensing circuit, and a low level communication controller could be powered down except for the brief period of communications with an implant unit. Low duty cycle of the transmission and reception would hold PIU 860 power consumption to a minimum.

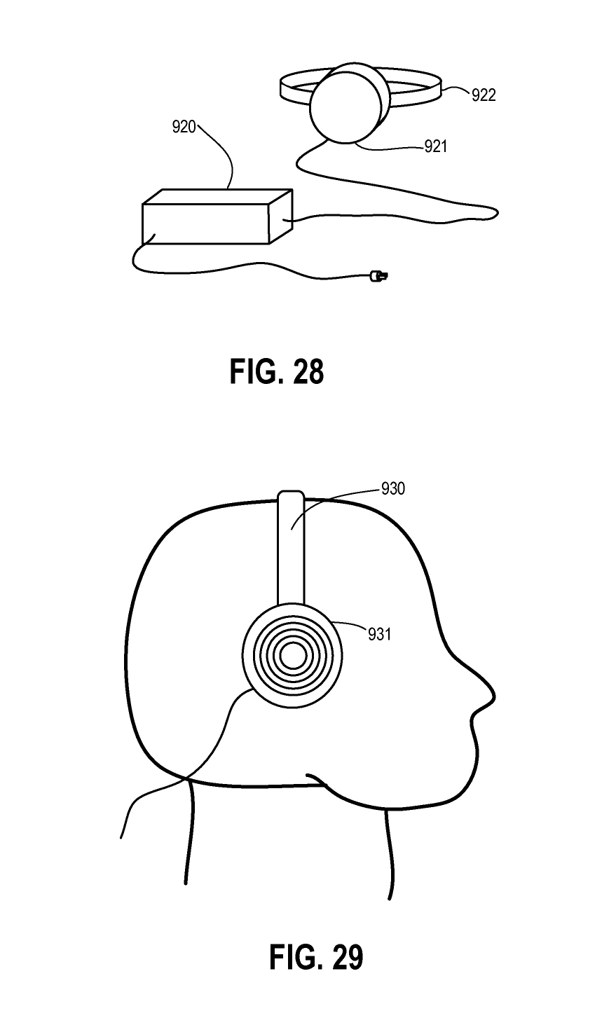

FIG. 28 shows a charging unit 920, according to some embodiments, for charging an implanted implant unit. Charging unit 920 is in some embodiments capable of charging an implant unit using an ergonomic method for locating the coil within the implanted unit for optimal power transmission through the electromagnetic interface, similar to such a feature described in connection with PIU 860. Charging unit 920 may also be capable of charging PIU 860 and/or downloading information stored in an implant unit being charged or in PIU 860. Charging unit 920 could then transmit downloaded information to a physician over a network link.

For embodiments of an implant unit that are implanted into the side of a patient's skull, coil 921 of charging unit 920 may be located on a device that fits behind the ear and secured with a strap 922. In other embodiments, coil 921 and a corresponding electronics and battery package may be incorporated into headphones or a pillow. FIG. 29 illustrates an external headset 930 which incorporates charging coil 931 into a portion that covers the ear. In some embodiments, charging unit 920 performs monitoring and/or programming functions similar to those performed by PIU 860. For example, some embodiments may include an external interface on headphones 930 (or on a computer or other device connected to headphones 930) permitting a patient or physician to turn an implanted VI pump on or off, select a delivery rate, and/or select a flow direction.

Several issues arise in the process of charging an implant unit battery. It is often desirable to nearly fully charge a battery at each charging session. A lithium ion (Li Ion) battery, for example, has an energy depletion curve has a large portion that is generally flat and at a nominal charge of approximately 3.3 volts. The curve drops off quickly near full depletion and spikes upward to slightly over 4 volts near full charge. Although it is desirable to charge as quickly as possible so as to reduce patient inconvenience, the rate of charging should be controlled. Overcharging an implant unit battery may cause damage, and battery life can optimized if the battery is only charged to a large fraction of full charge (i.e., not to one hundred percent). Overheating the battery during charging could cause tissue damage.

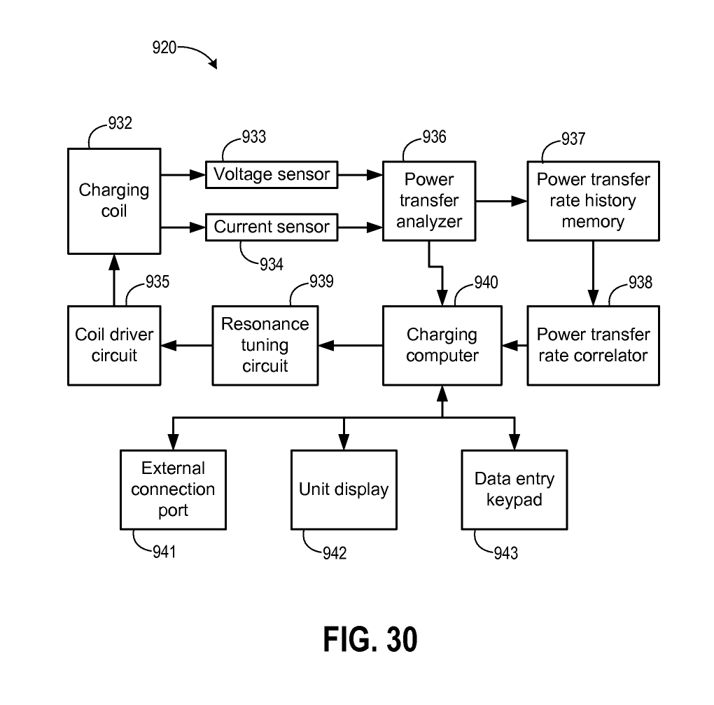

Measurement of implant unit battery voltage is useful when controlling charging. In order to minimize implant unit size and complexity, however, chargers according to some embodiments do not rely on an implant battery voltage monitoring circuit during battery charging. Instead, such chargers include circuitry that determines voltage, and thus charge level, in an implant unit battery. FIG. 30 is a block diagram of charging unit 920 according to some such embodiments. Charging unit 920 will produce a time varying magnetic field that will induce a current in the coil of an implant unit. Charging unit 920 will also monitor the voltage and current across and through a charging unit 920 coil in real time and calculate the energy transfer by evaluating the phase relationship. User interface controls on the charging unit can advise an operator regarding the transfer rate. With this information, the operator can adjust the placement and alignment between the charging unit 920 coil and the implant unit coil to optimize charging rate. Charging unit 920 can also maintain a data base of charging rates that is updated with usage. This information can be used to evaluate a coupling coefficient (described below), assuming the implant unit battery is able to absorb energy from the magnetic field of the charging unit 920 coil. If the implant unit battery is fully charged and the implant unit has shut down charging, the voltage and current in the charging unit 920 coil will remain orthogonal and charging unit 920 can notify the operator with a visual or audible alarm and/or shutdown.

Charging coil 932 produces the magnetic field that couples to the implant unit coil so as to transfer energy for charging the implant unit battery. Charging coil 932 (which may be implemented as coil 921 of FIG. 28 or coil 931 of FIG. 29) may be part of a resonant circuit, either series or parallel. The magnetic field may be produced with an inductor and drive circuit only. One model for the inductive coupling circuit of coil 932 to an implant unit coil (if secondary effects of series winding resistance and capacitance are ignored) is an ideal transformer with one side having a (1−K)*L inductor in series with a coil of the ideal transformer and a K*L inductor in parallel with that same ideal transformer coil, where K is the coupling coefficient and L is a primary inductance value. As K approaches 1, the coupling circuit appears as the ideal transformer in parallel with an inductor of value L. The coupling coefficient K will vary with placement and orientation of charging coil 932 relative to the coil of the implant unit. If the implant unit is near the skin, if coil 932 and the implant unit coil are parallel with their centers nearly aligned, and if the diameter of coil 932 is large compared to the distance between coil 932 and the implant unit coil, the coupling coefficient K will be high. Variation in coupling coefficient will be small.

Voltage sensor 933 and current sensor 934 are used to determine the phase relationship between the voltage across and the current through coil 932 so as to determine the amount of power being transferred. These sensors may be implemented in may different ways, including but not limited to pickoff coils, hall effect sensors, sense resistors, differential amplifies or other methods. Coil 932 is driven by coil driver circuit 935. In a resonant circuit, energy is transferred between the magnetic field energy and a capacitor voltage. During each cycle, the energy in the capacitor is converted into energy in the magnetic field and then back into capacitor energy. Some of the energy in the magnetic field is lost and is replenished to keep the oscillation going. This energy can be added in many different ways. It is common to add a small amount of voltage when the voltage magnitude is minimal or a small amount of current when the current is minimal. Other schemes are could be used.

Power transfer analyzer 936 monitors magnitude and phase of the voltage and current and calculates the total energy transfer. Energy lost during the charge cycle is absorbed by winding resistance, eddy currents produced in nearby conductors, and energy transferred to the implant unit coil and then to the implant battery charging circuitry. The total amount of energy taken from the resonant circuit can be calculated with knowledge of the voltage and current in that circuit. Most of the energy absorbers that contribute to energy loss are constant and can be eliminated from the calculations with historic information.

When an implant unit battery reaches full charge, a characteristic change in the energy transfer rates can be observed as the voltage increases above the nominal level. To detect this characteristic change, history of the energy transfer must be evaluated. This data is stored in a power transfer rate history memory 937. Power transfer rate correlator 938 is used to determine when the implant unit battery is nearing completion of its charging cycle. Many factors can cause fluctuation in the energy taken out of the resonant circuit, including temperature changes, orientation of coil 932 relative to an implant unit coil, distance between the coils, etc. It can be important that charging is not shut down prematurely, and that the battery is not overcharged. Power transfer rate correlator 938 looks for a specific pattern in the change in transfer rate. This pattern will vary in a predictable way with the rate of energy transfer and the type of battery being charged. With knowledge of the power curve for the implant unit battery and the transfer rate scale factors, correlator 938 estimates when the implant unit battery energy level is leaving the linear portion of its depletion curve and nearing completion of the charging cycle (e.g., when a Li Ion implant unit battery is nearing the spike in charge voltage corresponding to full charge). A goal may be detect when the battery charge level reaches near ninety percent and shut off at that time. Correlator 938 makes that determination and alerts charging computer 940.

Many factors cause small changes to the inductance of charging coil 932. To compensate for these changes, resonance tuning circuit 939 dithers the coil 932 driver frequency and successively makes small changes in the resonant capacitor to find and maintain the optimal resonant frequency.

Charging computer 940 evaluates input from correlator 938 as well as data from power transfer analyzer 936 to determine if shutdown is appropriate. Computer 940 also determines the appropriate amplitude of the magnetic field for proper operation and controls the level of coil driver circuit 935. Charging computer 940 also interfaces with a user through a key pad 943, a display 942 and an external computer connection port 941. For example, some implementations may require charging unit 920 to interface with other computers to transfer data, set parameters or download stored data, which operations may occur through port 941. A user of charging unit 920 may in some applications require charging status information and/or receive visual and/or audio queues about the charging status. Unit display 942 can provide such data output capabilities, including, but not limited to visual, audible, vibration or other forms of notification. As a further example, a simple implementation of charging unit 920 may require that it have the capability of starting a charging operation when commanded by an operator. Some systems may also require other commands to be executed when keys are pressed. Keypad 943 facilitates these functions.

In some embodiments, and as previously indicated, a physician controls PIU 860 using physician interface software executing on a conventional PC or other computer that is connected (e.g., by USB cable and/or a docking cradle) to PIU 860. Using the physician interface software, a physician can access and alter locked parameters stored within PIU 860. Such parameters can include limits on drug pump frequency and duty cycle, delivery time schedule, delivery frequency, ID number of implant devices that can be controlled with the PIU, duration of recorded data, calibration constants, etc.. The physician's interface software can require a password and the PIU ID to access the PIU key parameter memory. The physician's interface software can also download and/or delete the operational history file stored in the PIU. This operational file history can include, e.g., recharge times and duration, drug delivery duty cycle, communication error frequency, etc. Firmware and software within the PIU can also be updated via physician interface software. The physician interface software may also be able to download stored information in the pump such as usage data. The physician interface software will also allow enabling or disabling of certain patient controls (e.g., the ability to place an implant unit into hibernation or standby mode).

In some embodiments, PIU 860 will have a unique identification number used by the physician interface software to identify a specific PIU 860 and/or a patient assigned to that specific PIU. The software may also maintain patient data, nominal operating parameters and advice on limits appropriate for the application. The interface between the physician's interface software and PIU 860 can be password protected.

### Medical Uses of System

An implant unit according to one or more of the previously described embodiments can be implanted in a patient's skull behind the ear, where a pocket can be created within the mastoid bone. In some embodiments, additional implant units housing other implanted sub-system components can also be located in this pocket, e.g., a battery and electronics package (if not contained in a VI pump housing) and/or drug reservoir, with flexible catheter used to deliver the therapeutic agent to the target tissue (e.g. inner/middle ear, eye, brain, or other nervous system tissue). In some embodiments, the VI pump implant unit is small enough to be implanted within an eye or cochlea, with the control elements outside of the eye or cochlea. A multilumen tube can connect the eye- or cochlea-implanted pump unit with a vehicle source and the control electronics, with vehicle traveling through one lumen and control wires passing through another lumen.

Many patients with neurological disorders can benefit from a combination of electrical stimulation and drug delivery. In some embodiments, implanted drug delivery sub-systems such as are described above also include electronics and electrodes for electrical stimulation of the target tissue. Examples of catheters for local drug delivery and electrical stimulation are described in commonly-owned U.S. patent application Ser. No. 11/850,156.

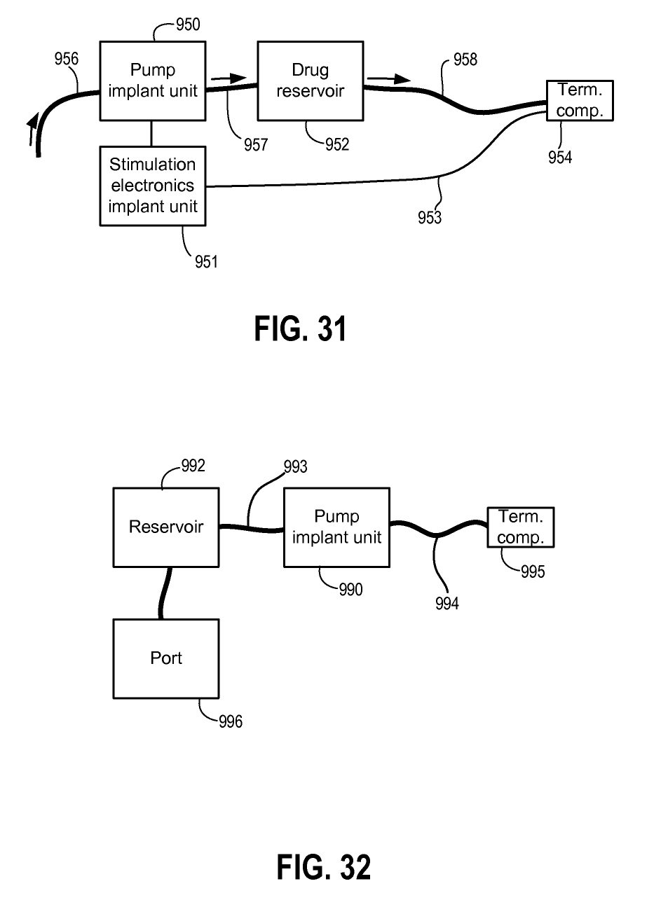

Terminal components for providing electrical stimulation in combination with targeted drug delivery can be used with any of the above described embodiments to treat a variety of target tissues. As one example, an implanted drug delivery sub-system such as is shown in FIG. 31 may include an implanted pump unit 950, implanted drug reservoir 952 and an implanted stimulation electronics package 951, with pump implant 950 receiving vehicle via catheter 956 and pumping that vehicle via catheter 957, reservoir 952 and catheter 958 to a terminal component 954. Terminal component 954 further includes an electrode receiving electrical signals from electronics 951 via wire 953. In other embodiments, pump implant 950, reservoir 952 and/or stimulation electronics 951 can be combined into a single implant unit. Numerous tissues can benefit from electrical stimulation. For example, inner or middle ear tissues can receive such a benefit. Electrical stimulation of the cochlear round window or promontory has been known to suppress tinnitus in some patients. Alternatively, a catheter delivering drugs into the inner ear may be combined with an electrode array such as those used for restoring hearing. As another example, and as described in commonly-owned application Ser. No. 11/780,853, a terminal component can be a retinal (or other intraocular) implant providing electrical stimulation and delivering drug-containing vehicle. As other examples, electrical stimulation and drug delivery may be used to treat the tissues of the deep brain (e.g., a treatment of Parkinson's disease), spine (e.g., a pain management), or inferior colliculus or auditory cortex for tinnitus or hearing related diseases. Deep brain stimulation may be used in conjunction with drug delivery for treatment of chronic pain states that do not respond to less invasive treatments. In some implementations, electrodes may be implanted in the somatosensory thalamus or the periventricular gray region. In some cases, the drug delivery system and implanted electrical stimulator may be located in two separate locations in the body. For example, stroke rehabilitation patients who receive electrical stimulation in their extremities (e.g., forearm or legs) to restore motor function may also receive plasticity-enhancing drugs in the brain (e.g. motor cortex) via an implanted drug delivery system.

Some additional embodiments include modification of one of the previously-described implantable systems to include a flow-rate sensor and a feedback loop to ensure that the actuating frequency is driving the desired flow-rate. Other embodiments may include a pressure sensor or a biosensor with output to a feedback loop or user display. In one example of a biosensor, an electrode may be used to detect round window noise as an indicator of tinnitus, and provide feedback to the pump to deliver a therapeutic agent to the inner ear or inferior colliculus accordingly. Still other embodiments may employ other types of sensors to provide biological feedback to the system.

In yet further embodiments, a VI pump can be run in the forward direction to deliver drug and in the reverse direction to either remove fluid from the selected tissue, reduce tissue pressure or to remove something else from the tissue. One example where such application might be helpful is in treatment of glaucoma. The VI pump can be operated in a reverse flow manner to remove fluid from the eye and then in a forward flow manner to deliver a drug to the eye that reduces the secretion of excess replacement fluid. Hydrocephalus (brain) is another condition in which it is useful to remove fluid pressure and deliver drug locally.

There are numerous circumstances in which it may be desirable to deliver drugs or other agents in a tissue-specific manner, on an intermittent or continuous basis and using one of the implantable drug delivery systems such as are described herein, to treat a particular condition. Disorders of the middle and inner ear may be treatable using systems and methods described herein. Examples of middle and inner ear disorders include (but are not limited to) autoimmune inner ear disorder (AIED), Meniere's disease (idiopathic endolymphic hydrops), inner ear disorder associated with metabolic imbalances, inner ear disorder associated with infections, inner ear disorder associated with allergic or neurogenic factors, blast injury, noise-induced hearing loss, drug-induced hearing loss, tinnitus, presbycusis, barotrauma, otitis media (acute, chronic or serious), infectious mastoiditis, infectious myringitis, sensorineural hearing loss, conductive hearing loss, vestibular neuronitis, labyrinthitis, post-traumatic vertigo, perilymph fistula, cervical vertigo, ototoxicity, Mal de Debarquement Syndrome (MDDS), acoustic neuroma, migraine associated vertigo (MAV), benign paroxysmal positional vertigo (BPPV), eustachian tube dysfunction, cancers of the middle or inner ear, and infections (bacterial, viral or fungal) of the middle or inner ear. Degenerative ocular disorders may also be treatable using systems and methods described herein. Examples of such degenerative ocular disorders include (but are not limited to) dry macular degeneration, glaucoma, macular edema secondary to vascular disorders, retinitis pigmentosa and wet macular degeneration. Similarly, inflammatory ocular diseases (including but not limited to birdshot retinopathy, diabetic retinopathy, Harada's and Vogt-Koyanagi-Harada syndrome, iritis, multifocal choroiditis and panuveitis, pars planitis, posterior scleritis, sarcoidosis, retinitis due to systemic lupus erythematosus, sympathetic ophthalmia, subretinal fibrosis, uveitis syndrome and white dot syndrome), ocular disorders associated with neovascularization (including but not limited to age-related macular degeneration, angioid streaks, choroiditis, diabetes-related iris neovascularization, diabetic retinopathy, idiopathic choroidal neovascularization, pathologic myopia, retinal detachment, retinal tumors, and sickle cell retinopathy), and ocular infections associated with the choroids, retina or cornea (including but not limited to cytomegalovirus retinitis, histoplasma retinochoroiditis, toxoplasma retinochoroiditis and tuberculous choroiditis) and ocular neoplastic diseases (including but not limited to abnormal tissue growth (in the retina, choroid, uvea, vitreous or cornea), choroidal melanoma, intraocular lymphoma (of the choroids, vitreous or retina), retinoblastoma, and vitreous seeding from retinoblastoma) may be treatable using devices and methods described herein.

Further examples of conditions that may be treatable using devices and methods described herein include, but are not limited to, the following: ocular, inner ear or other neural trauma; disorders of the auditory cortex; disorders of the inferior colliculus (by surface treatment or injection); neurological disorders of the brain on top of or below the dura; chronic pain; hyperactivity of the nervous system; migraines; Parkinson's disease; Alzheimer's disease; seizures; hearing related disorders in addition to those specified elsewhere herein; nervous disorders in addition to those specified elsewhere herein; ophthalmic disorders in addition to those specified elsewhere herein; ear, eye, brain disorders in addition to those specified elsewhere herein; cancers in addition to those specified elsewhere herein; bacterial, viral or fungal infections in addition to those specified elsewhere herein; endocrine, metabolic, or immune disorders in addition to those specified elsewhere herein; degenerative or inflammatory diseases in addition to those specified elsewhere herein; neoplastic diseases in addition to those specified elsewhere herein; conditions of the auditory, optic, or other sensory nerves; sensory disorders in additions to those specified elsewhere herein; conditions treatable by delivery of drug to the vicinity of the pituitary, adrenal, thymus, ovary, testis, or other gland; conditions treatable by delivery of drug to the vicinity of the heart, pancreas, liver, spleen or other organs; and conditions treatable by delivery of drug to specific regions of the brain or spinal cord.

The preceding identification of conditions is not intend to be an exhaustive listing. Drug delivery devices according to embodiments described herein can be used to deliver one or more drugs to a particular target site so as to treat one or more of the conditions described above, as well as to treat other conditions. As discussed above, many embodiments employ a drug capsule to dispense a drug that is in solid form. In some embodiments, however, a liquid or gel formulation can be used with a device whose drug reservoir can be refilled from the outside with a transcutaneous injection through a drug port. Drugs that can be delivered using implantable drug delivery systems such as are described herein include, but are not limited to, the following: antibiotics (including but are not limited to an aminoglycoside, an ansamycin, a carbacephem, a carbapenum, a cephalosporin, a macrolide, a monobactam, and a penicillin); anti-viral drugs (including but not limited to an antisense inhibitor, fomiversen, lamivudine, pleconaril, amantadine, and rimantadine); anti-inflammatory factors and agents (including but not limited to glucocorticoids, mineralocorticoids from adrenal cortical cells, dexamethasone, triamcinolone acetonide, hydrocortisone, sodium phosphate, methylprednisolone acetate, indomethacin, and naprosyn); neurologically active drugs (including but not limited to ketamine, caroverine, gacyclidine, memantine, lidocaine, traxoprodil, an NMDA receptor antagonist, a calcium channel blocker, a GABA_{A} agonist, an α2δ agonist, a cholinergic, and an anticholinergic); anti-cancer drugs (including but not limited to abarelix, aldesleukin, alemtuzamab, alitretinoin, allopurinol, altretamine, amifostine, anastrolzole, anti-hormones such as Arimidex®, azacitidine, bevacuzimab, bleomycin, bortezomib, busulfan, capecitabine, carboplatin, carmustine, chlorambucil, cisplatin, cyclophosphamide, cyclosporine, darbepoetin, daunorubicin, docetaxel, doxorubicine, epirubicin, epoetin, etoposide, fluorouracil, gemicitabine, hydroxyurea, idarubicin, imatinib, interferon, letrozole, methotrexate, mitomycin C, oxaliplatin, paclitaxel, tamoxifen, taxol and taxol analogs, topothecan, vinblastine and related analogs, vincristine, and zoledronate); fungicides (including but not limited to azaconazole, a benzimidazole, captafol, diclobutrazol, etaconazole, kasugamycin, and metiram); anti-migraine medication (including but not limited to IMITREX®); autonomic drugs (including but not limited to adrenergic agents, adrenergic blocking agents, anticholinergic agents, and skeletal muscle relaxants); anti-secretory molecules (including but not limited to proton pump inhibitors (e.g., pantoprazole, lansoprazole and rabprazole) and muscarinic antagonists (e.g., atropine and scopalomine)); central nervous system agents (including but not limited to analgesics, anti-convulsants, and antipyretics); hormones and synthetic hormones in addition to those described elsewhere herein; immunomodulating agents (including but not limited to etanercept, cyclosporine, FK506 and other immunosuppressant); neurotrophic factors and agents (factors and agents retarding cell degeneration, promoting cell sparing, or promoting new cell growth); angiogenesis inhibitors and factors (including but not limited to COX-2 selective inhibitors (e.g., CELEBREX®), fumagillin (including analogs such as AGM-1470), and small molecules anti-angiogenic agents (e.g., thalidomide)); neuroprotective agents (agents capable of retarding, reducing or minimizing the death of neuronal cells)(including but not limited to N-methyl-D-aspartate (NMDA) antagonists, gacyclidine (GK11), and D-JNK-kinase inhibitors); and carbonic anhydrase inhibitors (including but not limited to acetazolamide (e.g., DIAMOX®), methazolamide (e.g., NEPTAZANE®), dorzolamide (e.g., TRUSOPT®), and brinzolamide (e.g., AZOPT®)).

A variety of release systems may be used in connection with various combinations of the above identified (or other) drugs. The choice of the appropriate system will depend upon rate of drug release required by a particular drug regime. Degradable release systems may be used. Examples of degradable release systems include polymers and polymeric matrices, non-polymeric matrices, or/and organic excipients and diluents. Release systems may be natural or synthetic, though synthetic release systems are generally more reliable, more reproducible and produce more defined release profiles. The release system material can be selected so that drugs having different molecular weights are released from a particular cavity by diffusion through or degradation of the material. Embodiments of the invention include drug release via diffusion or degradation using biodegradable polymers.

In at least some embodiments, an implanted drug delivery system such as is described herein is used to deliver a drug (including but not limited to one or more of the drugs listed above) as a pure drug nanoparticle and/or microparticle suspension, as a suspension of nanoparticles and/or microparticles formed from drug formulated with binders and other ingredients to control release, or as some other type of nanoparticle- and/or microparticle-bound formulation. Nanoparticle- and/or microparticle-based delivery is advantageous in closed loop embodiments by allowing drug-containing particles to circulate within the closed loop as a solid suspended in the vehicle while delivering the desired therapeutic dose to the target tissue through the semi-permeable membrane or hollow fiber. Nanoparticle- and/or microparticle-bound delivery also offers the advantage of maintaining drug stability and avoiding loss of drug to polymeric components that may be encountered in a fluid pathway. Examples of nanoparticle drug formulations (and by extension, microparticle formulations) are described in commonly-owned U.S. patent application Ser. No. 11/831,230, which application is incorporated by reference herein.

Many diseases and disorders are associated with one or more of angiogenesis, inflammation and degeneration. To treat these and other disorders, devices according to at least some embodiments permit delivery of anti-angiogenic factors; anti-inflammatory factors; factors that retard cell degeneration, promote cell sparing, or promote cell growth; and combinations of the foregoing. Using devices described herein, and based on the indications of a particular disorder, one of ordinary skill in the art can administer any suitable drug (or combination of drugs), such as the drugs described herein, at a desired dosage.

Diabetic retinopathy is characterized by angiogenesis. At least some embodiments contemplate treating diabetic retinopathy by implanting devices delivering one or more anti-angiogenic factors either intraocularly, preferably in the vitreous, or periocularly, preferably in the sub-Tenon's region. It may also be desirable to co-deliver one or more neurotrophic factors either intraocularly, periocularly, and/or intravitreally.

Uveitis involves inflammation. At least some embodiments contemplate treating uveitis by intraocular, vitreal or anterior chamber implantation of devices releasing one or more anti-inflammatory factors. Anti-inflammatory factors contemplated for use in at least some embodiments include, but are not limited to, glucocorticoids and mineralocorticoids (from adrenal cortical cells).

Retinitis pigmentosa is characterized by retinal degeneration. At least some embodiments contemplate treating retinitis pigmentosa by intraocular or vitreal placement of devices secreting one or more neurotrophic factors.

Age-related macular degeneration (wet and dry) involves both angiogenesis and retinal degeneration. At least some embodiments contemplate treating this disorder by using one or more of the herein-described devices to deliver one or more neurotrophic factors intraocularly, preferably to the vitreous, and/or one or more anti-angiogenic factors intraocularly or periocularly, preferably periocularly, most preferably to the sub-Tenon's region.

Glaucoma is characterized by increased ocular pressure and loss of retinal ganglion cells. Treatments for glaucoma contemplated in at least some embodiments include delivery of one or more neuroprotective agents that protect cells from excitotoxic damage. Such agents include, but are not limited to, N-methyl-D-aspartate (NMDA) antagonists and neurotrophic factors. These agents may be delivered intraocularly, preferably intravitreally. Gacyclidine (GK11) is an NMDA antagonist and is believed to be useful in treating glaucoma and other diseases where neuroprotection would be helpful or where there are hyperactive neurons. Additional compounds with useful activity are D-JNK-kinase inhibitors.

Neuroprotective agents may be useful in the treatment of various disorders associated with neuronal cell death (e.g., following sound trauma, cochlear implant surgery, diabetic retinopathy, glaucoma, etc.). Examples of neuroprotective agents that may be used in at least some embodiments include, but are not limited to, apoptosis inhibitors, caspase inhibitors, neurotrophic factors and NMDA antagonists (such as gacyclidine and related analogs).

At least some embodiments may be useful for the treatment of ocular neovascularization, a condition associated with many ocular diseases and disorders and accounting for a majority of severe visual loss. For example, contemplated is treatment of retinal ischemia-associated ocular neovascularization, a major cause of blindness in diabetes and many other diseases; corneal neovascularization; and neovascularization associated with diabetic retinopathy, and possibly age-related macular degeneration.

A drug delivery device such as is described herein can be used to deliver an anti-infective agent, such as an antibiotic, anti-viral agent or anti-fungal agent, for the treatment of an ocular infection.

A drug delivery device such as is described herein can be used to deliver a steroid, for example, hydrocortisone, dexamethasone sodium phosphate or methylprednisolone acetate, for the treatment of an inflammatory disease of the eye.

A drug delivery device such as is described herein can be used to deliver a chemotherapeutic or cytotoxic agent, for example, methotrexate, chlorambucil, or cyclosporine, for the treatment of a neoplasm.

A drug delivery device such as is described herein can be used to deliver an anti-inflammatory drug and/or a carbonic anhydrase inhibitor for the treatment of certain degenerative ocular disorders.

Systems as described herein are especially useful for delivery of drugs to treat diseases that require continuous or frequent administration of a therapeutic over long periods of time (e.g., chronic, incurable conditions such as tinnitus or pain), and in which the treating drug may have serious side effects that make oral or parenteral administration unacceptable, or where the drug is more effective if combined with electrical stimulation. Systems such as described herein will permit the transport of a drug across barriers (such as the blood-brain barrier) that would not ordinarily be crossed by systemic drug administration.

Chronic infections located in a specific tissue and suppressible by long-term local treatment without developing resistance (e.g., viral infections) may be advantageously treated using systems such as are described herein.

The above list of treating drug and treated condition examples are merely illustrative and do not exclude uses of one or more other drugs in the previous list of example drugs to treat a condition in the previous list of example conditions.

### CONCLUSION

Certain embodiments are described above. The invention is not limited to the embodiments described above, and further includes (but is not restricted to) embodiments such as are described below.

For example, an implant unit similar to one or more of the above described embodiments could be used with a reservoir holding a liquid drug formulation and/or a pre-prepared drug nanoparticle suspension formulation. FIG. 32 shows one such embodiment. In FIG. 32, a pump implant unit 990 pumps liquid formulated drug from a reservoir 992, through catheters 993 and 994, to a terminal component 995. A separate implanted port 996 can be used to replenish drug in reservoir 992. In some embodiments, pump implant unit 990, reservoir 992 and/or port 996 could be combined into a single implant. In other embodiments, port 996 may be omitted and reservoir 992 may not be refillable.

Reservoir 992 may incorporate a collapsible housing of which the inner surfaces are in fluid communication with the port 996 and catheter 993. When liquid drug is injected into the port 996 to replenish the reservoir 992 the collapsible housing expands, and when the pump 990 draws fluid from the reservoir 992, the collapsible housing contracts. The outer surface of the collapsible housing is in fluid communication with body fluids that are external to the device which allows the pressure between the inside and outside of the housing to equalize, and thus passive expansion and contraction of the housing is possible. To prevent dosing of the patient during reservoir filling, a valve may be closed during filling, or the pump may be programmed to completely obstruct the fluid path as shown in FIG. 17B.

In additional embodiments a system may include more than one reservoir and/or pump for delivery of multiple drugs. In this case, the configuration shown in FIG. 31 (with or without the port) may be connected at the terminal end 995 to a system similar to FIG. 1 or FIG. 2. One or more reservoir/pump combinations delivering various drugs may be connected to the system in FIG. 1 at any location in the fluid path (e.g. catheter 7, 3, or 5), or FIG. 2 at any location in the fluid path (e.g. catheter 21, 23, or 25).

Embodiments of the invention include devices and systems that are configured for use in veterinary, diagnostic, laboratory, clinical research and development (“clinical R&D”) or other types of environments, as well as use of such devices and/or systems in such environments. For example, in systems intended for diagnostic, laboratory or clinical R&D environments, the pumping system and its associated control electronics may not be implanted (and if not implanted, may not be battery powered). A control device for such an embodiment may similarly have a different configuration (e.g., may not communicate wirelessly with the pump control electronics, may combine functions of the physician's programmer and PIU described above, may be in the same housing as the pump(s) and the pump-driving electronics, etc.). Embodiments intended for veterinary use may have different physical configurations and/or sizes corresponding to the size and type of animal in which the device is to be implanted, may not be implanted, may be configured to use an animal cage as an antenna, etc.

Some embodiments may only have a single catheter (or other fluid conduit) that penetrates the housing of implant unit. For example, the implant unit may contain liquid in a reservoir and include one or more valves to release the liquid upon command or in response to preprogrammed instructions. In still other embodiments, an implant unit may contain reservoirs holding multiple types of liquids (e.g., diagnostic reagents) that can be controllably released, with each reagent reservoir having a separate conduit (e.g., a separate catheter, a separate lumen of a multi-lumen catheter) for delivery to a target site. Such embodiments could include multiple pumps in the implant unit (e.g., multiple pumps on a chip), may be non-implantable, and/or may be configured for use in veterinary, diagnostic, laboratory, clinical R&D, or other environments.

In some embodiments a variety of sensors may be added, with the sensors used to detect various physiological indicators and instruct an implant unit to operate accordingly (e.g., turn on or off, deliver drug on detection of a particular chemical or electrical imbalance, etc.). In some embodiments, for example, a pressure sensor implanted within or near the eye could be used to detect excessive pressure and to activate an implant unit pump in order to relieve that pressure, and then to reverse the pump flow (by changing actuator frequency) to pump drug (after opening a valve from a drug chamber) into the eye to prevent more pressure build-up.

For embodiments employing wireless communication with an implanted pump, different frequencies, modulation types and data coding schemes can be employed. In some embodiments, a PIU may communicate with an implant unit via conventional RF signals.

Numerous characteristics, advantages and embodiments of the invention have been described in detail in the foregoing description with reference to the accompanying drawings. However, the above description and drawings are illustrative only. The invention is not limited to the illustrated embodiments, and all embodiments of the invention need not necessarily achieve all of the advantages or purposes, or possess all characteristics, identified herein. Various changes and modifications may be effected by one skilled in the art without departing from the scope or spirit of the invention. Although example materials and dimensions have been provided, the invention is not limited to such materials or dimensions unless specifically required by the language of a claim. The elements and uses of the above-described embodiments can be rearranged and combined in manners other than specifically described above, with any and all permutations within the scope of the invention.

1. An implant unit, comprising:
a pump chamber including a flexible wall, an inlet opening and an outlet opening;
a force-transferring member configured to compress the flexible wall at an actuation position, wherein the actuation position
generally defines a first sub-chamber located between the inlet opening and the actuation position and a second sub-chamber located between the outlet opening and the actuation position, and
is located such that compression of the flexible wall at the actuation location while a fluid is in the pump chamber results in a higher fluid pressure in the first sub-chamber relative to the second sub-chamber and a net fluid flow through the pump chamber; and
a housing sized for implantation in a living human or other animal and separating an internal space containing the pump chamber and the force-transferring member from an exterior, the housing including an external surface facing the exterior and formed from a biocompatible material, a first housing opening in fluid communication with the pump chamber inlet and a second housing opening in fluid communication with the pump chamber outlet.

2. The implant unit of claim 1, wherein
the pump chamber comprises a flexible conduit,
the pump chamber inlet opening and the pump chamber outlet opening comprise walls that are substantially less elastic than the flexible conduit, and
the actuation position is asymmetrically located between the inlet opening and the outlet opening.

3. The implant unit of claim 2, further comprising an electromagnet within the housing, and wherein
the force-transferring member comprises a magnetically-reactive material, and
the electromagnet and the force-transferring member are configured such that the force-transferring member compresses the flexible conduit when power is not applied to the electromagnet and such that compression of the flexible conduit is relieved when power is applied to the electromagnet.

4. The implant unit of claim 2, wherein the housing is elongated and has first and second ends, wherein the first and second housing openings are located at the first end of the housing, and further comprising
a drug reservoir located at the second housing end, the drug reservoir including an internal volume; and
a first fluid conduit placing the first housing opening in fluid communication with the drug reservoir internal volume along a first fluid path, and wherein
the second housing opening and the drug reservoir internal volume are in fluid communication along a second fluid path, and
the flexible conduit is part of the second fluid path.

5. The implant unit of claim 4, wherein the housing has an outer diameter that does not exceed 10 millimeters.

6. The implant unit of claim 2, wherein the force-transferring member comprises a magnetically-reactive material and is configured to compress the flexible conduit in response to a magnetic field originating from a source external to the implant unit.

7. The implant unit of claim 2, wherein the force-transferring member comprises a magnetically-reactive material, and further comprising electrically conductive windings surrounding the flexible conduit on opposite sides of the force-transferring member.

8. The implant unit of claim 2, further comprising
an electro-reactive actuating element within the housing configured to move the force-transferring member to compress the flexible conduit,
a sealed barrier dividing the housing internal space into a first internal space containing the flexible conduit and the force transferring member and a second internal space containing the electro-reactive actuating element.

9. The implant unit of claim 1, wherein the flexible wall comprises a flexible membrane.

10. The implant unit of claim 1, further comprising a drug reservoir in fluid communication with the pump chamber and containing at least one of a solid drug, a nanoparticle or microparticle mass, or a gel- or liquid-formulated drug.

11. The implant unit of claim 10, wherein the drug reservoir is attached to or contained within the housing.

12. The implant unit of claim 1, further comprising an electro-reactive actuating element configured to move the force-transferring member and at least one implant unit processor configured to activate the electro-reactive actuating element.

13. The implant unit of claim 12, further comprising at least one memory, and wherein the at least one implant unit processor is further configured to activate the electro-reactive actuating element according to multiple dosing sequences stored in the memory, each dosing sequence including a time at which fluid is to be pumped through the pump chamber.

14. The apparatus of claim 13, wherein each dosing sequence further includes a duty cycle corresponding to a number of times the force-transferring member is to be moved during the dosing sequence.

15. The apparatus of claim 14, wherein the at least one implant unit processor is further configured
to wirelessly communicate with at least one external device, and
to modify a dosing sequence stored in the memory in response to a received communication.

16. The apparatus of claim 15, wherein the at least one implant unit processor is further configured
to activate the electro-reactive actuating element in response to an instruction in a received instruction,
to store data corresponding to times at which the electro-reactive actuating element has been activated, and
to wirelessly communicate the stored data to an external device.

17. The apparatus of claim 15, further comprising a battery and a charging coil, and wherein the implant unit is configured to charge the battery using electrical energy output by the coil in response to an applied magnetic field, to receive communications by demodulating magnetic signals received by the coil, and to transmit communications using the coil.

18. The implant unit of claim 13, further comprising a patient interface unit, the patient interface unit having at least one patient interface unit processor configured to perform operations that include
wirelessly communicating instructions to the at least one implant unit processor, after the implant unit is implanted in a living human or other animal, causing activation of the implant unit, and
wirelessly communicating instructions to the at least one implant unit processor, after the implant unit is implanted in a living human or other animal, causing deactivation of the implant unit.

19. The implant unit of claim 18, wherein the patient interface unit comprises a coil, and wherein the patient interface unit is configured to generate a magnetic field with the coil sufficient to charge a battery of the implant unit after the implant unit has been implanted in a living human or other animal.

20. The implant unit of claim 18, wherein the at least one patient interface unit processor is configured to communicate with software executing on a computer separate from the patient interface unit.

21. The implant unit of claim 1, further comprising:
a battery;
a piezoelectric element configured to generate force in response to a drive voltage;
a plurality of voltage stages, each voltage stage configured to receive an input voltage and provide a higher output voltage, each voltage stage comprising a capacitor and a switch network configurable to alternately charge and discharge the capacitor according to a charge cycle for the stage, the voltage stages arranged in series to sequentially increase the input voltage so as to yield a drive voltage greater than a maximum voltage obtainable from the battery alone; and
a timing control sequence circuit configured to control switching of the voltage stage switch networks according to the respective charge cycles, wherein a charge cycle frequency of each voltage stage of the plurality after a first stage in the series is one half the charge cycle frequency of the immediately preceding voltage stage of the series.

22. The implant unit of claim 17, wherein the electro-reactive actuating element comprises a piezoelectric element configured to generate force in response to a drive voltage, and wherein the battery is connected to one side of the charging coil, and further comprising:
a charge capacitor; and
a voltage comparison and switch control circuit configured to, according to a constant duty cycle, alternately energize the charging coil with the battery and de-energize the charging coil so as to charge the charge capacitor.

23. An implant unit, comprising:
a housing sized for implantation into the body of a living human and having a biocompatible exterior;
a valveless impedance pump contained within the housing;
a drug reservoir, in fluid communication with the valveless impedance pump, containing a supply of solid drug removable by flow of vehicle passing through the valveless impedance pump and the drug reservoir;
a first fluid conduit in fluid communication with one of the valveless impedance pump and the drug reservoir;
a second fluid conduit in fluid communication with the other of the valveless impedance pump and the drug reservoir; and
a third fluid conduit in placing the valveless impedance pump in fluid communication with the drug reservoir.

24. The implant unit of claim 23, wherein the drug reservoir is contained in the housing.

25. The implant unit of claim 23, further comprising:
an actuator configured to cause compression of a flexible wall of a fluid chamber of the valveless impedance pump in response to an applied electrical power;
a battery;
a coil configured to output electrical energy in response to a magnetic field applied by an external source; and
control electronics configured to control the actuator, to control charging of the battery from the electrical energy output by the coil, and configured to receive communications from an external device via the coil.
